(19) United States
(12) Patent Application Publication
(10) Pub. No.: US 2019/0231936 A1
(43) Pub. Date: Aug. 1, 2019

(54) METHOD OF MAKING THROMBORESISTANT/BACTERICIDAL S-NITROSO-N-ACETYLPENICILLAMINE (SNAP)-IMPREGNATED NITRIC OXIDE RELEASE POLYMERS WITH ENHANCED STABILITY

(71) Applicant: Biocrede, Inc., Ann Arbor, MI (US)

(72) Inventor: Hao Chen, Ann Arbor, MI (US)

(73) Assignee: Biocrede, Inc.

(21) Appl. No.: 15/886,451

(22) Filed: Feb. 1, 2018

(51) Int. Cl. A61L 29/08; C01B 21/24; A61K 47/34; A61K 47/59; A61K 31/16; A61K 31/095; C08L 75/04; C08L 69/00; C08L 83/04

(52) U.S. Cl. CPC A61L 29/085; C01B 21/24; A61K 47/34; A61K 47/59; A61K 31/16; C08L 2203/02; C08L 75/04; C08L 69/00; C08L 83/04; C08J 2300/22; A61K 31/095

(57) ABSTRACT

A method for making an NO-releasing polymeric composition, a discrete RSNO adduct is dissolved in a solvent to form a discrete RSNO adduct solution. A polymer material is soaked in the discrete RSNO adduct solution for a predetermined time to swell the polymer material and impregnate the polymer material with the discrete RSNO adduct.

### STATEMENT REGARDING FEDERALLY SPONSORED RESEARCH OR DEVELOPMENT

This invention was made with government support under NIH-R41-DK100161-01, NIH-R41-DK101206-01, and NIH-R43-DK102189-01 awarded by the National Institutes of Health. The government has certain rights in the invention.

### BACKGROUND

Nitric oxide (NO), a gas molecule endogenously produced by various NO synthase (NOS) enzymes, has been shown to have several important physiological functions, including its unique vasodilating properties, cancer-fighting potency, antibacterial and/or anti-biofilm properties (e.g., inhibiting proliferation of bacteria, fungi and viruses), and anti-platelet activity. Although NO is a stable radical, it may be highly reactive with hemoglobin and oxygen, thus making delivery of NO to the target site challenging. Stable hydrophilic NO donors, as well as hydrophobic NO donors may be best to take advantage of the potency of NO for a wide range of biomedical applications. These applications include NO-releasing pharmaceuticals and the preparation of thromboresistive hydrophobic polymeric coatings for medical devices, such as intravascular catheters and extracorporeal circuits (based on NO's antiplatelet activity). However, despite the benefits of NO, the use of NO donors in polymeric systems has been relatively limited for various reasons, including storage stability, costly synthesis, and/or limited NO release lifetimes.

### BRIEF DESCRIPTION OF THE DRAWINGS

Features and advantages of examples of the present disclosure will become apparent by reference to the following detailed description and drawings, in which like reference numerals correspond to similar, though perhaps not identical, components. For the sake of brevity, reference numerals or features having a previously described function may or may not be described in connection with other drawings in which they appear.

FIG. 1A is a schematic cut-away view showing thrombus formation on a siloxane-based polyurethane elastomer (e.g., ELAST-EON™ E2As) control coated extracorporeal circulation (ECC) circuit;

FIG. 1B is a schematic cut-away view showing a S-nitroso-N-acetylpenicillamine (SNAP)/E2As coated ECC circuit, which releases nitric NO and reduces thrombus formation;

FIG. 2A shows the structure of S-nitroso-N-acetyl-D,L-penicillamine (SNAP);

FIG. 2B shows a scheme of S-nitrosothiol (RSNO) decomposition, which can be catalyzed by metal ions (e.g., Cu⁺), light, and heat, yielding the disulfide (RSSR) product and nitric oxide (NO);

FIGS. 3A and 3B show diffusion of SNAP from various polymer films containing 10 wt % SNAP, monitored at 340 nm, films were immersed in 4 mL phosphate buffered saline (PBS) in the dark at room temperature, 22° C. (FIG. 3A) or 37° C. (FIG. 3B) (the data is the mean±SEM (standard error of the mean) (n=3));

FIG. 4A shows NO release behavior of 10 wt % SNAP/E2As film at 37° C. in the dark, ambient light, and 100W floodlight (the data is the mean±SEM (n=3));

FIG. 4B shows NO release from 10 wt % SNAP in silicone rubber (SR), CARBOSIL®, and ELAST-EON™ E2As films at 37° C. and continuously irradiated with the 100W floodlight (the data is the mean±SEM (n=3));

FIG. 4C shows NO release from 5 wt % and 10 wt % SNAP in Elast-Eon™ E2As films at 37° C. continuously under ambient light (amb) or the 100W floodlight (the data is the mean±SEM (n=3));

FIG. 5A shows UV-vis spectra of a 10 wt % SNAP/E2As film, 1.0 mM SNAP, and E2As dissolved in N,N-dimethylacetamide (DMAc) (the data is the mean±SEM (n=3));

FIG. 5B shows cumulative NO release from 10 wt % SNAP/E2As films incubated in PBS under various conditions: room temperature (22° C.) with ambient light, 37° C. in the dark, 37° C. under ambient light, and 37° C. under the 100W floodlight (the data is the mean±SEM (n=3));

FIG. 6A shows diffusion of SNAP from 10 wt % SNAP-doped E2As films soaking in 1 mL PBS in the dark, as monitored at 340 nm, at room temperature (RT, 22° C.) or 37° C.;

FIG. 6B shows a comparison of the cumulative SNAP leaching and cumulative NO release (from NOA-based or SNAP-based NO release data) from the 10 wt % SNAP-doped E2As films soaking in PBS at 37° C. in the dark. Nitric oxide release from SNAP-doped E2As films can occur from thermal and/or photochemical decomposition of SNAP within the polymer phase, or from SNAP that leached into the aqueous phase. For the SNAP-doped E2As films, approximately 26% of the total NO release is attributed to the SNAP leaching;

FIG. 7 is a graph showing diffusion of SNAP from 10 wt % SNAP in E2As films with 0, 2, or 4 top coats of E2As as monitored at 340 nm by UV-vis, the films were soaked in 10 mM PBS containing 100M EDTA, which was replaced daily after the UV-vis measurement, at 37° C. in the dark (the data is the mean±SEM (n=3));

FIG. 8 is a graph showing stability of 10 wt % SNAP in E2As films stored dry with desiccant under various temperature and light conditions, the films were dissolved in DMAc to determine the amount of SNAP remaining at various times (compared to the initial level) as monitored at 340 nm by UV-vis (the data is the mean±SEM (n=3));

FIG. 9 is a graph showing NO release behavior from 10 wt % SNAP/E2As dry film at 37° C. in the dark;

FIG. 10 is a schematic diagram of the extracorporeal circuit (ECC) tubing coated with 5 wt % SNAP/E2As followed by a top coat of E2As;

FIGS. 11A and 11B are graphs showing representative NO surface flux profiles from a section of ECC tubing coated with 5 wt % SNAP in E2As before (FIG. 11A) and after (FIG. 11B) blood exposure, NO release was measured via chemiluminescence at 37° C. under ambient light;

FIGS. 12A and 12B are graphs showing time-dependent effects of the 5 wt % SNAP/E2As coating on platelet count (e.g., consumption) (FIG. 12A) and plasma fibrinogen levels (FIG. 12B) during the 4 hour blood exposure in the rabbit thrombogenicity model (the data is the mean±SEM (n=4));

FIG. 13 is a graph showing results of in vitro fibrinogen adsorption assays on the 5 wt % SNAP/E2As and E2As control coatings (fluorescence assay in a 96-well plate that used goat anti-human fibrinogen-FITC conjugated antibody to measure the level of adsorbed human fibrinogen (3 mg/mL) on the coatings, the data is the mean±SEM (n=24));

FIG. 14 is a two-dimensional representation of thrombus formation on the SNAP/E2As and control ECCs after 4 hour blood exposure in the rabbit thrombogenicity model, as quantified using ImageJ software from NTH (the data is the mean±SEM (n=4));

FIG. 15 is a graph showing an NO release profile of polyurethane tubing (a micro-polyurethane tubing available from Scientific Commodities, Inc.) impregnated with SNAP, the tubing having been soaked in a SNAP/acetone solution for either 1 or 2 days;

FIG. 16A is a schematic illustration of a catheter tubing coated with an active layer of 5 wt % or 10 wt % SNAP/E2As followed by a top coat of E2As;

FIG. 16B is a cross-sectional view of the catheter tubing of FIG. 16A, taken along line 16B-16B of FIG. 16A;

FIG. 17 shows NO release profiles of 5 wt % and 10 wt % SNAP/E2As catheters at 37° C. in the dark (n=5);

FIG. 18 is a graph showing quantitation of thrombus area on SNAP/E2As catheters and E2As control catheters after 7 day implantation in sheep, as calculated with NIH ImageJ software using a 2D representation of thrombus (the data are means±SEM (n=5)), *=p<0.05;

FIG. 19 is a comparison of bacterial adhesion (CFU/cm²) on 1 cm piece of explanted SNAP/E2As catheters and E2As control catheters (the data are means±SEM (n=5)), *=p<0.05;

FIG. 20A is a graph illustrating the release of NO from SNAP impregnated silicone Foley catheter tubings over a 30 day period (data are the mean±SEM (n=3));

FIG. 20B is a graph illustrating the NO release profile of one SNAP impregnated catheter segment after 3 days of soaking in phosphate buffered saline (PBS);

FIG. 21 is a graph illustrating the percentage of total loaded SNAP that leaches from the surface of the silicone tubing during the first 7 days of soaking in PBS at 37° C. (data are the mean±SEM (n=3));

FIGS. 22A and 22B are, respectively, plate count results and fluorescence images for S. epidermidis biofilms developed on Foley catheters segments in a CDC biofilm reactor for 3, 7 and 14 days, where FIG. 22A illustrates the plate count of the number of viable bacteria adhered to the SNAP impregnated and control catheter surfaces, and where FIG. 22B includes the black and white representations of representative fluorescence images of the SNAP impregnated and control catheter surfaces taken with an oil immersion 60× objective lens;

FIGS. 23A and 23B are, respectively, plate count results and fluorescence images for P. mirabilis biofilms developed on Foley catheters segments in a CDC biofilm reactor for 3, 7 and 14 days, where FIG. 23A illustrates the plate count of the number of viable bacteria adhered to the SNAP impregnated and control catheter surfaces, and where FIG. 23B are the black and white representations of representative fluorescence images of the SNAP impregnated and control catheter surfaces taken with an oil immersion 60× objective lens or with a 10× objective lens (the latter of which was used for the 14 day control catheter only);

FIGS. 24A and 24B are, respectively, plate count results and fluorescence images for P. mirabilis formation on SNAP impregnated and control (no SNAP) Foley catheter segments after 3 days in CDC reactor for catheter segments that were first soaked in PBS buffer for 24 hours before the start of the biofilm formation experiments, to reduce any initial higher flux of NO from the outer surface of the SNAP-impregnated catheter segments, where FIG. 24A illustrates the plate count of the number of viable bacteria adhered to the SNAP impregnated and control catheter surfaces, and where FIG. 24B includes the black and white representations of representative fluorescence images of the SNAP impregnated and control catheter surfaces taken with an oil immersion 60× objective lens; and

FIG. 25 is a graph illustrating the nitric oxide flux of a SNAP impregnated catheter segment after 3 days of P. mirabilis biofilm growth in a CDC biofilm reactor (n=3), where the catheter pieces had been pre-soaked in PBS buffer for 24 hours prior to the start of the experiment, where the 96 hour and 120 hour time-points correspond to the day of cell viability assessment and the following day.

### DETAILED DESCRIPTION

Examples according to the present disclosure include a novel method of making RSNO-doped polymer formulation useful for making biomedical devices. In some examples, the novel polymer formulations form homogeneous films and exhibit RSNO stability even at 37° C. for 4 months (with only about a 10%-15% loss of NO).

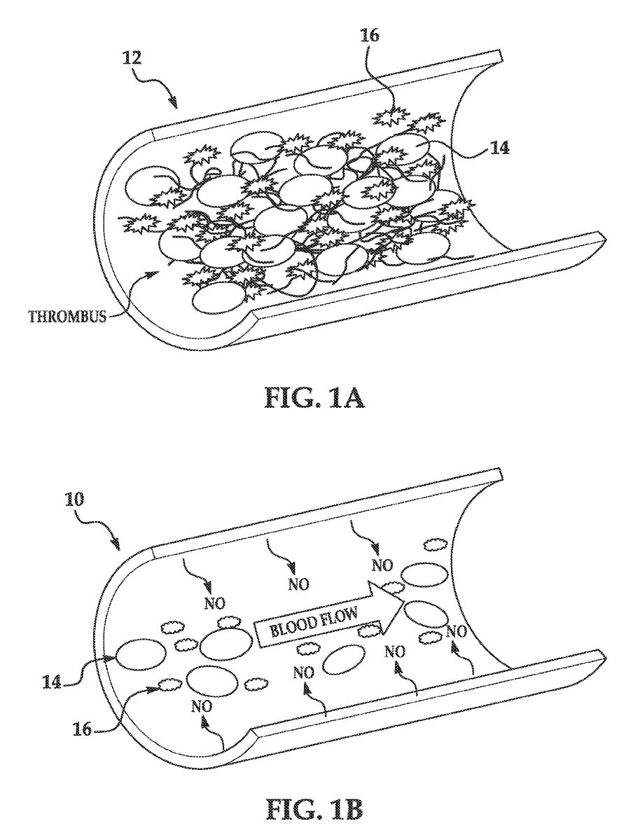

In some examples, the polymer formulations may be formulated and used as coatings to prevent thrombus (i.e., blood clot) formation in, e.g., extracorporeal circulation (ECC) circuits. FIG. 1A is a schematic cut-away view showing a siloxane-based polyurethane elastomer (e.g., Elast-Eon™ E2As) control coated ECC circuit 12 that exhibits thrombus formation. As illustrated, the red blood cells 14 and platelets 16 clot together. In contrast, FIG. 1B is a schematic cut-away view showing an example 10 according to the present disclosure of a S-nitroso-N-acetylpenicillamine (SNAP)-doped siloxane-based polyurethane elastomer (e.g., SNAP/E2As) coated ECC circuit that does not exhibit thrombus formation. As depicted in FIG. 1B, NO is generated, which contributes to the red blood cells 14 and platelets 16 not clotting together.

In some other examples, the polymer formulations may be formulated using the novel method by dissolving the SNAP (or other suitable discrete RSNO adduct) in a suitable solvent to form a solution, and then soaking a polymer (e.g., an existing low water uptake biomedical polymer) in the solution. Soaking is allowed to occur for a predetermined time (e.g., at least 24 hours) to swell the polymer material and impregnate the polymer material with the discrete RSNO adduct. Examples of the polymer include silicone rubber, siloxane-based polyurethane elastomers, thermoplastic silicone-polycarbonate-urethane, etc., and examples of the solvent include tetrahydrofuran, chloroform, methylene chloride, cyclohexanone, and combinations thereof. The swelling/impregnation method disclosed herein is particularly suitable for introducing SNAP into Foley catheters (i.e., indwelling urinary catheter formed of a flexible tube and having two separated lumens). In an example, the swelling/impregnation method is conducted at room temperature (e.g., from about 17° C. to about 24° C.) with commercially available Foley catheters, and therefore avoids exposing the SNAP to elevated temperatures that are often associated with catheter extrusion processes. As such, in the swelling/impregnation method disclosed herein, the SNAP is not exposed to elevated temperatures that would render it unstable and cause it to prematurely decompose. Several advantages have been observed with SNAP-impregnated Foley catheter tubing, such as a steady release of NO for one month at physiological and antimicrobial levels, and a decrease in the degree of microbial biofilm formation after 14 days of exposure to flowing media inoculated with P. mirabilis or Staphylococcus (S.) epidermidis, two bacterial strains that most often induce catheter-associated urinary tract infections (CAUTIs).

Blood/material interaction is important to the success of implantable medical devices, ranging from simple catheters, stents and grafts, to complex extracorporeal artificial organs that are used in thousands of patients every day. Thrombosis is one of the primary problems associated with clinical application of blood contacting materials. Despite a thorough understanding of the mechanisms of blood-surface interactions and decades of bioengineering research effort, the ideal non-thrombogenic prosthetic surface remains an unsolved problem. Over the last 50 years, much has been learned about surface-induced thrombosis and attempts to prevent it with systemic anticoagulation and surface modifications. Surface modifications have included using pure, very smooth silicone rubber or polyurethane, pre-exposure of the surfaces to albumin and other coating proteins, and surface binding of heparin in an ionic as well as a covalent fashion. Despite extensive research to develop a non-thrombogenic surface that mimics the endothelium, none of these modifications have been successful.

Nitric oxide (NO) has been found to be one of two potent vasodilators secreted by normal endothelium that has the ability to inhibit platelet adhesion/activation and aggregation to the blood vessel wall. The NO-flux from a normal and stimulated endothelium has been estimated to be in the range of 0.5×10⁻¹⁰ mol cm⁻² min⁻¹ to 4×10⁻¹⁰ mol cm⁻² min⁻¹. Nitric oxide has been extensively studied for its inhibitory effects on circulating platelet and monocyte activation that leads to aggregation and ultimately initiation of thrombosis. A wide range of NO donors such as S-nitrosothiols (RSNOs), N-Hydroxy-N-nitrosoamines, N-diazeniumdiolates and nitrosyl metal complexes have been studied at least over the past decade.

Nitric oxide (NO) can be released from an NO adduct/donor species appended to polymers within a polymer coating. “Nitric oxide adducts” (NO adducts) and “NO-donors” refer to compounds and functional groups which, under physiological conditions, can donate and/or release NO such that biological activity of the NO is expressed at the intended site of action/target site.

Some examples according to the present disclosure include the NO donor/adduct within a polymer coating. The NO donor/adduct may be integrated into the polymer coating in any suitable manner, examples of which include doping and swelling/impregnating. Suitable NO adducts (examples of which include discrete adducts) are generally those exhibiting capability of embedding (either by covalent attachment and/or dispersion) into the polymer matrix and exhibiting process preparation stability.

“Discrete NO adducts” as referred to herein are those NO adducts (examples of which are RSNOs) which, when placed into a polymer matrix, release therapeutically relevant fluxes of NO, ranging from about 0.2 ×10⁻¹⁰ mol cm⁻² min⁻¹ to about 20 ×10⁻¹⁰ mol cm⁻² min⁻¹ of NO from the polymer phase. Those compounds that have their NO-releasing moiety covalently attached to a polymer backbone are generally referred to as “polymeric NO adducts.” Examples of suitable polymeric NO adducts include, but are not limited to, S-nitrosothiolated polyurethanes, S-nitrosothiolated silicone rubbers, and/or mixtures thereof. Some examples of the discrete NO adducts exhibit some lipophilicity, but may be made more lipophilic by derivatization with one or more alkyl groups.

As such, examples of the present disclosure are novel methods for making nitric oxide (NO) releasing formulations formed from polymers impregnated with S-nitroso-N-acetylpenicillamine (SNAP) to prevent thrombus formation in, e.g., extracorporeal circulation (ECC) circuits and catheter tubing.

Various hydrophobic polymer materials may be employed in examples of the material, method, and device as disclosed herein. These include, but are not limited to materials such as polyurethanes (PU), silicone rubbers (SR), copolymers of polyurethane and silicone rubber (e.g., E2A), poly(vinyl chloride) (PVC), polymethacrylates, polyacrylates, polycaprolactones, and/or mixtures thereof. In other examples, the polymer material may include both hydrophobic and hydrophilic domains. The polymer of choice will be one capable of releasing NO from, for example, covalently attached and/or dispersed S-nitrosothiol (RSNO) type NO-adducts within the polymer. The polymer of choice may also depend upon the application in which polymer coating/film will be used and the desired NO release rate for that application. As examples, a polymer having higher water uptake may be suitable in applications where quick NO release is desirable, while a polymer having lower water uptake may be suitable in applications were slow NO release is desirable. In instances where prolonged NO release is desirable, poly(lactic-co-glycolic acid) (PLGA) additives may also be included in the polymer coating/film to create an acidic environment to further stabilize the RSNO species.

Further, a system is contemplated as being within the purview of the present disclosure that includes discrete RSNOs doped into a polymer, with the polymer also having RSNO appended thereto (e.g., by covalent attachment). For example, previously prepared polyurethane polymers with appended RSNO functional groups can be mixed with discrete RSNOs or similar species to create the long-term NO release polymers enabled by the present disclosure.

In some examples, the NO adduct of choice is one capable of spontaneous release of NO when the polymer is exposed to solutions and/or blood under physiological conditions. In other examples, the NO adduct of choice is one capable of spontaneous release of gas phase NO when the polymer is exposed to certain light conditions. Some examples of NO adducts include discrete S-nitrosothiols (RSNOs).

It is believed that examples of the present disclosure including SNAP doped into siloxane-based polyurethane elastomers (one example of which is E2As) may help stabilize the RSNO adduct, thus advantageously allowing longer NO release from the RSNO species and enhanced storage stability, even at higher temperatures (e.g., 37° C.).

Spontaneous release of NO from the polymer may be governed by at least one process occurring between the NO adduct and the surrounding environment. For RSNO species, these include, but are not limited to temperature, moisture, and the presence of certain wavelengths of light. For example, photolysis of the S-N bond in the RSNO species liberates NO gas. Photolysis can occur with light in either the 300 nm to 400 nm wavelength range or the 500 nm to 600 nm wavelength range. In this example, the efficiency of NO release is generally greater in the higher wavelength range.

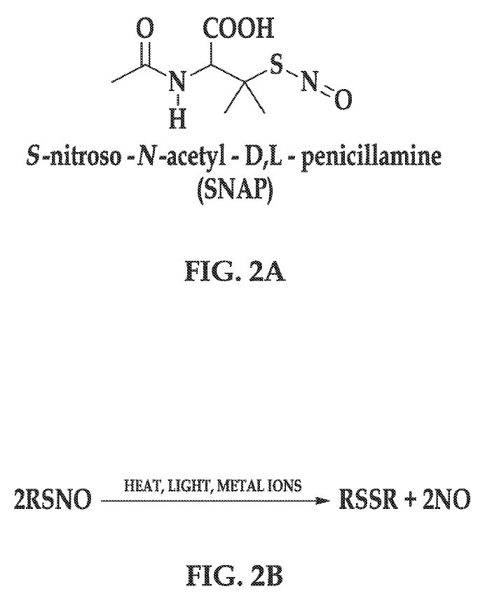

It is to be understood that discrete nitric oxide adducts may be either covalently attached to the polymer matrix or may be dispersed therein, or both. Some examples of discrete RSNOs include, but are not limited to S-nitrosoglutathione (GSNO), S-nitroso-N-acetylpenicillamine (SNAP, shown in FIG. 2A), S-nitrosocysteine (CysNO), etc., and derivatized discrete RSNOs. Derivatized RSNOs may be modified with alkyl group(s). As examples, a derivative may have an alkyl group attached to the free carboxyl group of SNAP and/or may have a longer alkyl (i.e., longer than acetyl) attached to the amine group of S-nitrosopenicillamine. As an example, an ester linkage may be formed between the desired alkyl group and the free carboxyl group of SNAP. As another example, a long chain alkyl (including from 4 to 10 carbon atoms) may replace the acetyl group of SNAP so that the long chain alkyl is attached to the amine nitrogen. As other examples, a sugar may be attached to the carboxyl group of SNAP (e.g., glucose-SNAP, mannose-SNAP, fructose-SNAP, etc.).

The SNAP-doped NO release siloxane-based polyurethane elastomer coatings according to examples of the present disclosure were evaluated in vitro and within a short-term in vivo rabbit model of thrombogenicity. The coatings according to examples of the present disclosure continuously released from 0.5 to 1 ×10⁻¹⁰ mol cm⁻² min⁻¹ NO for 20 days in the dark, soaking at 37° C. in PBS. Additionally, the novel coatings retained about 78% of the SNAP after 4 months at 37° C. in the dark (i.e., not exposed to wavelengths that could photolyze RSNO bonds) and in dry conditions (i.e., in the presence of a desiccant). As discussed further herein, examples of the coating materials were employed as inner wall coatings of extracorporeal circuits used for 4 hours of extracorporeal circulation (ECC) in a rabbit model of thrombogenicity to examine the effect of the coatings on platelet function, clotting and fibrinogen adsorption. The SNAP-doped NO release coatings were also used to fabricate catheters, which were implanted in sheep veins for 7 days to evaluate the effects on thrombus and bacterial adhesion.

The SNAP impregnated catheter tubings made according to the novel method were also evaluated in vitro. These tubings exhibited significant anti-biofilm properties using two bacteria strains (S. epidermidis and P. mirabilis) that are responsible for high rates of nosocomial urinary catheter associated infections.

As mentioned above, nitric oxide (NO) is an endogenous gas molecule that plays several key physiological roles, including prevention of platelet adhesion and activation, inhibiting bacterial adhesion and proliferation, enhancing vasodilation, promoting angiogenesis, and aiding in wound healing. The effects of NO are highly dependent on the location of the NO and its concentration in the physiological system. For example, endothelial cells that line the inner walls of healthy blood vessels produce an estimated NO surface flux ranging from 0.5 mol cm⁻² min⁻¹ to 4.0×10⁻¹° mol cm⁻² min⁻¹. The function of many blood-contacting devices, including vascular grafts, stents, intravascular sensors, intravascular catheters, and extracorporeal life support circuits, can be impaired due to platelet activation and thrombus formation. One approach to improve the hemocompatibility of such devices is the use of coating materials that mimic the endothelial cells with respect to NO release. Indeed, in recent years there has been considerable interest in developing NO-release and NO-generating materials that can be used to improve the biocompatibility of such devices.

Nitric oxide also exhibits antimicrobial activity, including killing bacteria and preventing biofilm formation. Bacterial infections and biofilm formation are problems that can cause complications with biomedical devices. Bacteria also possess the ability to form biofilms on surfaces when the organism secretes a polysaccharide matrix in which the bacteria will live. This matrix provides both nutrients and protection against the host defense and antibiotics. Biofilms can act as a source of chronic infection, thereby prolonging the recovery time. Among its many biological roles, nitric oxide functions as an antimicrobial agent and as an accelerant to the wound healing process. Nitric oxide has broad-spectrum antibacterial properties, killing both gram-positive and gram-negative bacteria. Low levels of nitric oxide are also reported to efficiently disperse biofilms that have formed on the surface of indwelling medical devices.

Nitric oxide is highly reactive under physiological conditions, and thus a wide range of NO donor molecules with functional groups that can store and release NO have been studied for potential biomedical applications. Such molecules include organic nitrates, metal-NO complexes, N-diazeniumdiolates, and S-nitrosothiols (RSNOs). Physiological RSNOs, such as S-nitrosohemoglobin and S-nitrosoglutathione (GSNO), are considered an endogenous reservoir of NO in vivo. Other synthetic RSNOs, such as S-nitroso-N-acetyl-L-cysteine (SNAC) and S-nitroso-N-acetylpenicillamine (SNAP, FIG. 2A) have been shown to exhibit significant antimicrobial and antithrombotic effects. It has also been demonstrated that RSNOs are both vasodilators and potent inhibitors of platelet aggregation. RSNOs undergo thermal decomposition, releasing NO and producing a corresponding disulfide species (RSSR), as shown in FIG. 2B. The NO release from RSNOs can be catalyzed by metal ions (e.g., Cu⁺) and by light, through the irradiation at energies that correspond to the S-nitroso absorption bands at 340 nm and/or 590 nm. It has been suggested that the more potent activity of RSNOs vs. NO as antiplatelet agents arises from the enhanced stability of RSNOs vs. NO, and generation of NO from RSNOs locally at the surface of platelets by membrane proteins that contain catalytic sites to convert RSNO to NO.

Incorporation of RSNOs into polymers can extend the utility of these NO donors to be applicable as coatings in biomedical devices, providing localized NO release at the blood/device interface. Several NO-release polymers consisting of small-molecule RSNOs dispersed in various polymer matrices, including polyethylene glycol (PEG), poly(vinyl alcohol), poly(vinyl pyrrolidone), and PLURONIC® F127 hydrogel, have been suggested. These materials have potential applications for topical NO delivery on wounds via the diffusion of the hydrophilic RSNOs from the polymer to the tissue. In fact, daily application of a GSNO-containing hydrogel has been shown to accelerate the wound healing process. However, the rapid leaching of the RSNOs from such polymers can significantly shorten the NO/RSNO release lifetime, lasting only several hours. An alternate approach has been to synthesize RSNO-modified materials, where the RSNO functionality is covalently bound to the matrix. Fumed silica particles, dendrimers, polyurethanes, polyesters, polydimethylsiloxane (PDMS), xerogels, self-assembled monolayers, and poly(vinyl methyl ether-co-maleic anhydride) (PVMMA) have all been modified with RSNO functionalities. RSNO-modified xerogels were found to release NO for up to 14 days and exhibit reduced platelet and bacterial adhesion. However, such RSNO-modified xerogels suffer from synthesis complications leading to cracking and non-uniform films, low RSNO conversion efficiency (maximum of 40% for the tertiary RSNO-modified xerogels), and thermal instability at room temperature that would limit their shelf-life. Many of the other RSNO modified materials reported to date exhibit both thermal and photoinitiated NO release, but many of these materials have not proven clinically useful due to their limited NO release lifetimes or lack of the RSNO functionality stability during storage, or low conversion to RSNO during synthesis.

Another approach reported to achieve localized NO delivery at a polymer/blood interface is to use NO-generating coatings, in which immobilized catalysts (Cu(I/II) or organoselenium species) can generate NO from endogenous RSNOs. For example, a NO generating coating containing Cu⁰ nanoparticles was evaluated recently using a rabbit model for extracorporeal circulation (ECC). However, to achieve good efficacy in reducing thrombus formation, continuous infusion of SNAP was required to supplement the endogenous RSNO levels.

In order to avoid the continuous infusion of RSNO species, the present disclosure includes several biomedical polymers that are capable of storing RSNO species. The RSNO-impregnated polymers made according to the to the novel method can advantageously release NO, as well as potentially supplement the endogenous RSNO levels, if NO generating catalysts are also employed.

To further illustrate the present disclosure, examples are given herein. It is to be understood that these examples are provided for illustrative purposes and are not to be construed as limiting the scope of the present disclosure.

### EXAMPLE 1

In this example of the present disclosure, five biomedical grade polymers doped with S-nitroso-N-acetylpenicillamine (SNAP) were investigated for their potential to control the release of NO from the SNAP within the polymers, and further control the release of SNAP itself. As discussed further herein, SNAP is quite stable in the ELAST-EON™ E2As polymer, creating a homogeneous coating that can locally deliver NO (via thermal and photochemical reactions) as well as slowly release SNAP. E2As is an example of suitable siloxane-based polyurethane elastomers contemplated as being within the purview of the present disclosure. E2As is a solution grade of E2A (see Table 1 below). The E2As polymer containing SNAP was coated on the walls of extracorporeal circuits (ECC) and exposed to 4 hour blood flow in a rabbit model of extracorporeal circulation to examine the effects on platelet count, platelet function, clot area, and fibrinogen adsorption. After 4 hours, platelet count was preserved at 100±7% of baseline for the SNAP/E2As coated loops, compared to 60±6% for E2As control circuits (n=4). The SNAP/E2As coating also reduced the thrombus area when compared to the control (2.3±0.6 and 3.4±1.1 cm², respectively). As will be discussed further herein, the SNAP/E2As catheters were also able to significantly reduce the thrombus area and bacterial adhesion after 7 day implantation in sheep veins. All of the results suggest that the new SNAP/E2As coatings have potential to improve the thromboresistance of intravascular catheters, grafts, and other blood contacting medical devices.

Five biomedical polymers (silicone rubber (SR), ELAST-EON™ E2As (a siloxane-base polyurethane elastomer commercially available from Aortech Biomaterials, Scoresby Victoria, Australia), CARBOSIL® (a thermoplastic silicone-polycarbonate-urethane commercially available from DSM Biomedical Inc., Berkeley, Calif.), TECOFLEX™ SG80A and TECOPHILLIC™ SP-60D-60 (both polyurethanes commercially available from The Lubrizol Corporation, Wickliffe, Ohio)) were examined for their potential to act as a storage reservoir for SNAP. The ELAST-EON™ polymer has excellent intrinsic biocompatibility and biostability properties, and exhibits low levels of blood protein adsorption. Each of the SNAP-doped polymers is characterized for its in vitro NO/SNAP release. It was found that SNAP itself is stable for at least 4 months in the ELAST-EON™ E2As polymer, creating a coating that releases NO thermally (at physiological temperature) and can also serve as a reservoir to supplement endogenous RSNO levels (by SNAP diffusion into blood from the polymer). The SNAP/E2As polymer was tested for potential biomedical applications via, e.g., an ECC rabbit model of thrombogenicity to assess preservation of platelet count and function, and thrombus area after 4 hours of ECC.

It is to be understood that other siloxane-based polyurethane elastomers (aside from E2As) are also contemplated as being suitable for use in the present disclosure. Further, it is to be understood that other grades of ELAST-EON™ siloxane-based polyurethane elastomers (commercially available from Aortech Biomaterials, Scoresby Victoria, Australia) are contemplated as being suitable for use in the present disclosure. Table 1 below is a table of properties of various grades of ELAST-EON™ polymers. In addition to those examples shown in Table 1, it is believed that other suitable polymers include CARBOSIL®, PURISIL™, or silicone rubber.

TABLE 1
Elast-Eon ™ Properties

| TEST | E5-130 | E5-325* | E2A* | E2-945 | E2-852 | E2-860 | E2-862 | E4 |
|---|---|---|---|---|---|---|---|---|
| Durometer hardness | 77A | 82A | 90A | 50D | 55D | 65D | 68D | 80D |
| Tensile Strength, MPa | 21 | 23 | 26 | 28 | 30 | 34 | 34 | 60 |
| Elongation at Break, % | >700 | >500 | >450 | >400 | >300 | >200 | >200 | 25 |
| Tensile Stress, 100% E, | 4 | 5 | 8 | 12 | 15 | 23 | — | — |
| MPa | | | | | | | | |
| Tensile Stress, 200% E, | 5 | 7 | 10 | 15 | 18 | — | — | — |
| MPa | | | | | | | | |
| Tensile Stress, 300% E, | 6 | 9 | 13 | 18 | 23 | — | — | — |
| MPa | | | | | | | | |
| Modulus of Elasticity, MPa | 11 | 15 | 35 | 115 | 360 | 650 | 650 | — |
| Tear Strength, kN/m | 45 | 60 | 80 | 97 | 129 | — | — | — |
| Physical Form | Pellets | Pellets** | Pellets** | Pellets | Pellets | Pellets | Pellets | Pellets |
| Melt Temperature ° C. | 175-185 | 180-185 | 195-200 | 220 | 210-215 | 210-215 | 210-215 | 185 |

*Can also be supplied in solution grade and is soluble in both THF and DMAc.
**Also supplied in dispersion form.

Preliminary In vitro Characterization of Various SNAP-Doped Polymer Films for Example 1

SNAP doped into all of the five biomedical polymers produced homogeneous and transparent films of green color, without any observable phase separation. The 10 wt % SNAP films stored approximately 0.42 μmol of SNAP per mg polymer film (or 6 μmol/cm²), while the 5 wt % SNAP films stored approximately 0.21 μmol of SNAP per mg polymer film (or 3 μmol/cm²).

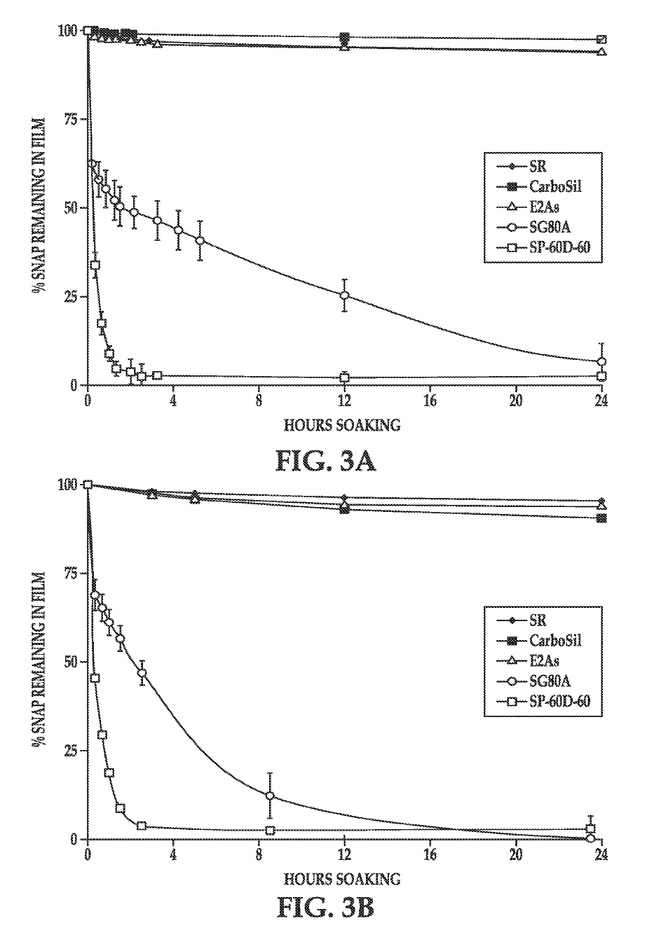

The 5 wt % and 10 wt % SNAP/polymer films were immersed in 4 mL PBS in the dark at room temperature (i.e., 22° C.) or at 37° C. The diffusion of SNAP into the PBS from the various polymer films containing 5 wt % and 10 wt % SNAP was monitored using UV-Vis absorption. As shown in FIGS. 3A and 3B, all of the SNAP diffused out of the SG80A and SP-60D-60 polymer films during the first day of soaking in PBS at room temperature (see FIG. 3A) and at 37° C. (see FIG. 3B). The SP-60D-60 polymer is hydrophilic with a water uptake of about 60 wt %, while the SG80A is more hydrophobic, having a water uptake of about 6 wt % (see Table 2 below).

Table 2 illustrates the water uptake of the 5 biomedical polymers used in the present disclosure. Polymer films (200 mg polymer) were cast in Teflon® ring (d=2.5 cm) on Teflon® plates. Small disks (d=0.7 cm) were cut from the parent films, weighed, and immersed in PBS for 48 hours at 37° C. The wet films were wiped dry and weighed again. The water uptake of the polymer films is reported in Table 2 in weight percent as follows: water uptake (wt %)=(W_{wet}-W_{dry})/W_{dry}×100, where W_{wet} and W_{dry} are the weights of the wet and dry films, respectively.

TABLE 2

| Polymer | Water uptake [wt %] |
|---|---|
| Silicone Rubber | 1.2 ± 0.3 |
| CarboSil | 1.5 ± 0.3 |
| Elast-Eon ™ E2As | 1.2 ± 0.1 |
| Tecoflex SG80A | 6.2 ± 0.7 |
| Tecophilic SP-60D-60 | 64.5 ± 0.1 |

As shown in FIGS. 3A and 3B, all of the SNAP leaves the more hydrophilic SP-60D-60 polymer during the initial 2 hours of soaking, while the more hydrophobic SG80A leaches all of the SNAP after 24 hours. Due to the rapid loss of the SNAP from the SP-60D-60 and SG80A polymers, a very large initial burst of NO was observed via chemiluminescence (with a Nitric Oxide Analyzer (NOA)) during the first day of soaking (Day 0), and the films exhibited no SNAP/NO release after one day (data not shown). Therefore, these two polymers provide a quick burst of NO/SNAP and were found not to be suitable for longer-term release of NO/SNAP.

In contrast, the silicone rubber, CARBOSIL®, and E2As polymers exhibit significantly lower amounts of SNAP diffusing into the soaking buffer after one day (see FIGS. 3A and 3B). For all three of these polymers, an initial burst of SNAP leaching was observed during the first day of soaking, corresponding to rapid water uptake by the polymer. This initial burst was about 10% of the total SNAP molecules incorporated into the films. Small amounts of SNAP continued to leach from these polymers during the subsequent days of soaking. Silicone rubber, CARBOSIL® (a thermoplastic silicone-polycarbonate-urethane), and E2As (a siloxane-base polyurethane elastomer) all are hydrophobic polymers due to their high PDMS content and also have the lowest water uptake (see Table 1 above). SNAP is slightly hydrophobic. Therefore, SNAP should have a preference for remaining in these more hydrophobic polymer films. In addition, it is believed that the hydrophobic property of these polymers also plays a significant role in limiting the diffusion of SNAP into the buffer, due, at least in part, to the minimal water uptake of these polymers.

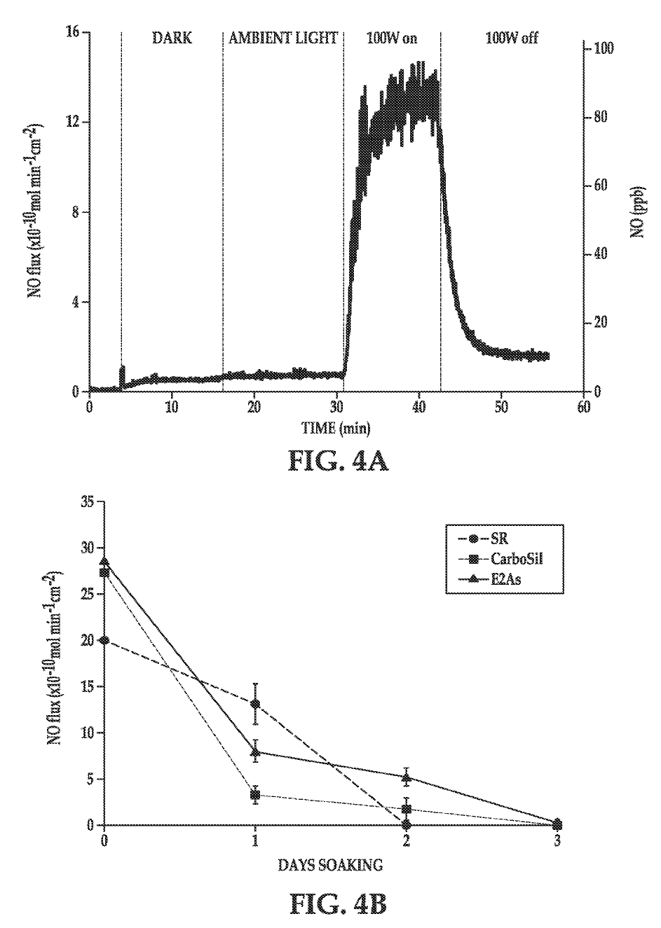

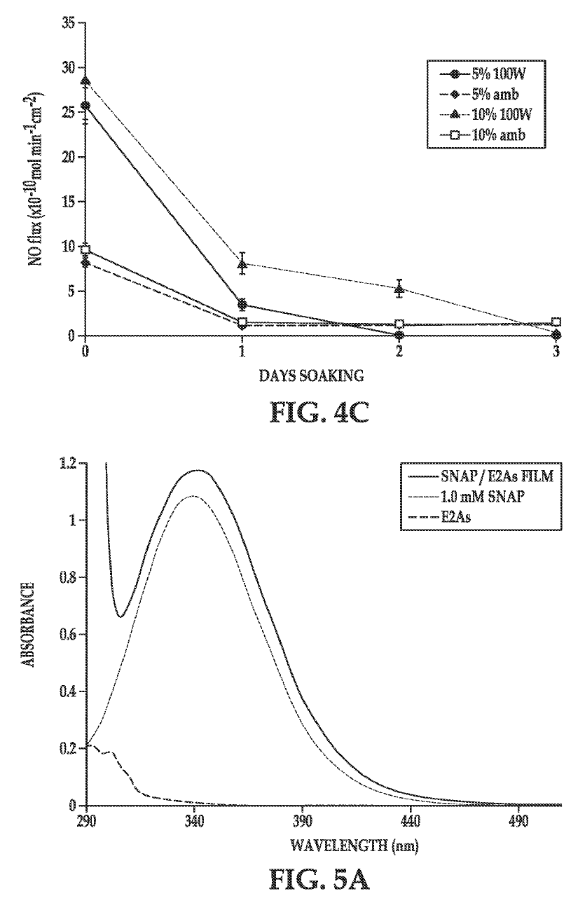

The thermal and photoinitiated NO release from the three SNAP-doped polymers (i.e., silicone rubber, CARBOSIL®, and E2As polymers) was also studied by NOA measurements. Nitric oxide release can be turned on/off using the 100W floodlight for all 3 film types. FIG. 4A illustrates the NO release behavior of the 10 wt % SNAP/E2As film at 37° C. in the dark, in the ambient light, and in the 100W floodlight. As shown in FIG. 4A, there is little difference in the NO release from the 10 wt % SNAP/E2As film in the dark or under the ambient lab lights, since ambient fluorescent lighting does not emit the wavelengths responsible for decomposing RSNOs (340 nm or 590 nm). While the data for the 10 wt % SNAP/E2As film is shown, it is to be understood that for all three polymers, the total NO release detected by the NOA for films continuously irradiated with the 100W floodlight was about 100% of the SNAP doped into the films. The photoinitiated NO release from these three films was examined by continuously irradiating with a 100W floodlight at 37° C. and monitoring the NO released with the NOA (see FIG. 4B). The SNAP-doped E2As and CARBOSIL® films exhibited a gradual decrease in the photo-induced NO flux over a 3 day period, while the SR-based films released NO for only 2 days under the same conditions. All three types of films incubated at 37° C. under ambient light yielded an initial burst of NO on the first day of soaking, corresponding to release of SNAP into the solution, and on subsequent days, the NO flux ranged from 1 ×10⁻¹⁰ mol cm⁻² min⁻¹ to 2 ×10⁻¹⁰ mol cm⁻² min⁻¹. This NO flux is still potentially useful to inhibit platelet function and kill bacteria.

It appears that the NO release may be more promising from the film composed of 10 wt % SNAP in E2As under the 100W floodlight. Therefore, the wt % of SNAP in E2As was varied to 5 wt % and examined in more detail (see FIG. 4C). The NO release and SNAP leaching pattern is similar for the 5 wt % SNAP/E2As film, but the NO release takes place over a shorter time period. The biostability and biocompatibility of the Elast-Eon™ polymers in combination with the NO release from SNAP makes this formulation attractive for further in vitro and possible biomedical applications.

Long-Term NO Release of SNAP/E2As Formulation for Example 1

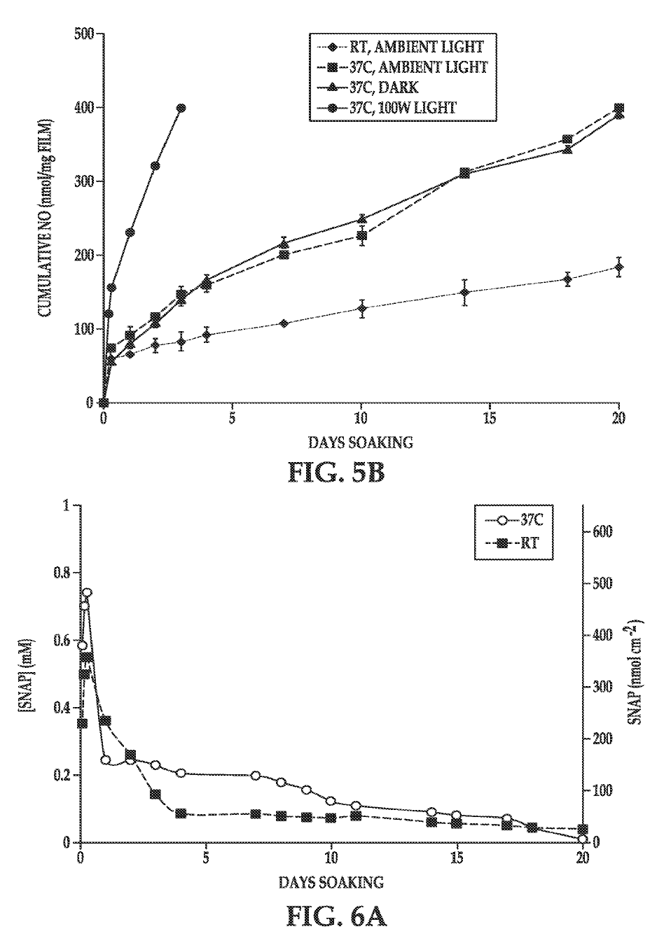

In vitro studies were conducted with the SNAP/E2As films to examine the long-term NO release and SNAP leaching from these films. The NO release from the SNAP/E2As films over time was determined based on the amount of SNAP decomposed within the polymer phase (i.e., by measuring the SNAP remaining after dissolving the films at given time points). The initial concentration of SNAP in the 10 wt % films was 420 nmol SNAP/mg film. FIG. 5A shows the UV-Vis spectra of 1.0 mM SNAP solution, a 10 wt % SNAP in E2As film redissolved in N,N-dimethylacetamide (DMAc), and E2As dissolved in DMAc.

Due to thermal and/or photochemical decomposition of SNAP, a decrease in the 340 nm absorbance band was observed as the 10 wt % SNAP/E2As films were soaked in PBS under various conditions, and the cumulative NO release based on that absorbance decrease is shown in FIG. 5B. The 10 wt % SNAP/E2As films displayed an initial burst of NO during the first day of soaking (see FIGS. 3A and 3B), which corresponds to the thermal decomposition as well as diffusion of SNAP out of the film. Films soaked at room temperature had the lowest flux of NO release. However, films incubated at 37° C. in the dark or under ambient light exhibited a higher NO release than the films at room temperature. This is due to the increased thermal decomposition of SNAP. The films that were exposed to ambient light yield essentially the same NO release profiles as the films that were soaked in the dark. Nitric oxide release from the SNAP/E2As films that arc continuously irradiated with the 100W floodlight at 37° C. only release NO for 3 days due to their higher NO fluxes that rapidly deplete the SNAP reservoir. These films provide NO release via both a thermal and photoinitiated decomposition of SNAP.

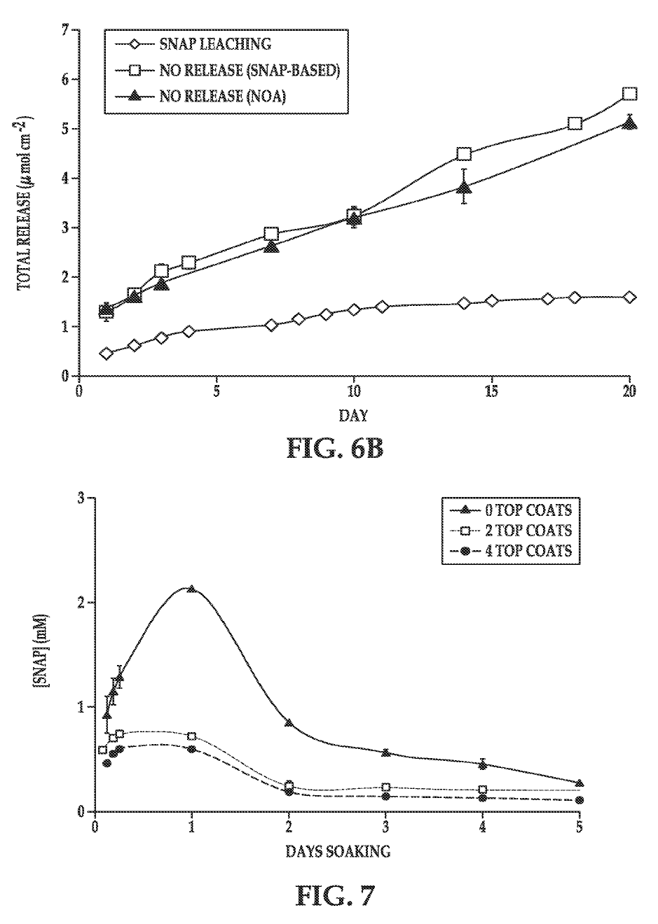

Nitric oxide release from the SNAP-doped E2As can occur from thermal and/or photochemical decomposition of SNAP either within the polymer phase, or after SNAP enters the aqueous phase by diffusion out of the polymer. In order to better understand the NO release mechanism of the SNAP/E2As coating, the SNAP diffusion into PBS was monitored over a 20 day period. As shown in FIG. 6A, the films containing 10 wt % SNAP at 37° C. exhibit an initial burst of SNAP leaching on the first day. After this initial burst, the SNAP continues to slowly diffuse from the E2As until the SNAP reservoir is nearly depleted (with still measurable amounts of SNAP leaching on day 20). The total moles of SNAP that leach from the film accounts for about 27% of the total NO released (as detected by NOA measurements) during the 20 day period (see FIG. 6B).

Additionally, the effect of the number of polymer top coats on loss of SNAP was also evaluated. SNAP-doped E2As films without any top coat exhibit higher levels of SNAP diffusion into the buffer than films with at least 2 topcoats (see FIG. 7). The thickness of the top coat allows control of the diffusion rate of SNAP from the polymer reservoir. As such, in the examples disclosed herein the SNAP-doped films may be coated with a polymer top coat. Examples of suitable polymers for the top coat include the siloxane-based polyurethane elastomers, poly(vinyl chloride), crosslinked polyurethanes, crosslinked silicone rubber, polytetrafluoroethylene, etc. (without the NO donor therein). In some examples, the polymer used for the top coat is the same polymer used for the underlying film.

Stability Study of the SNAP/E2As Films for Example 1

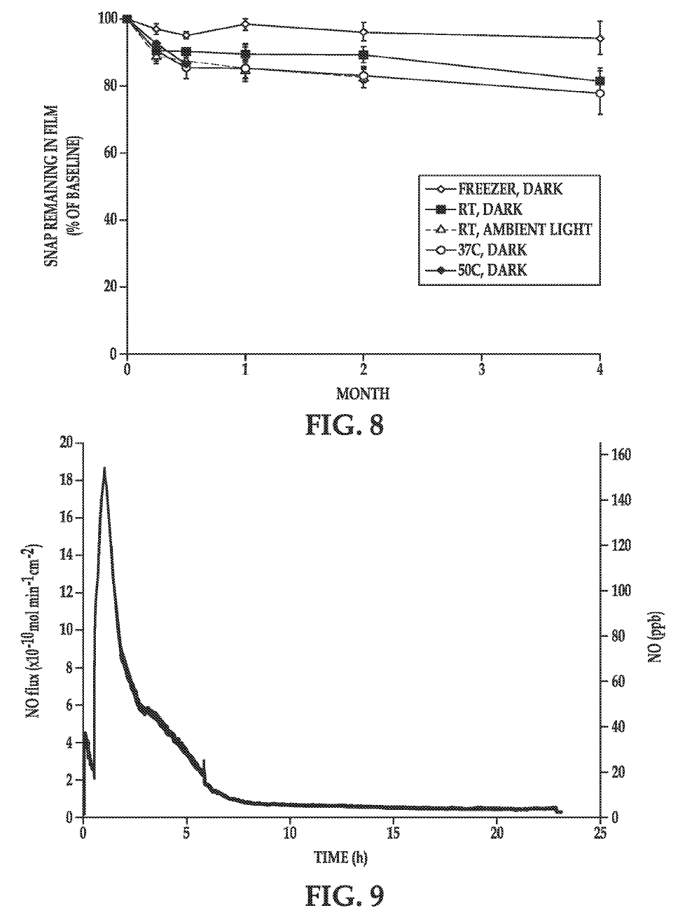

The stability of SNAP doped in the E2As polymer during dry storage was also evaluated. SNAP incorporated in E2As can potentially undergo thermal or photochemical decomposition during storage, thus limiting the available NO release capacity at the time of use. Therefore, 10 wt % SNAP/E2As films were stored with desiccant in the freezer in the dark, dry in the dark or in ambient light with desiccant at room temperature, and dry in the dark with desiccant at 37° C. and 50° C. These stability studies were conducted in a similar manner as the cumulative NO release experiments, where films were dissolved in DMAc to determine the amount of SNAP remaining in the polymer at various time points (as described herein). The results indicate that SNAP is stable within the E2As polymer matrix after 4 months. After 2 months, for example, the 10 wt % SNAP films stored in the freezer (−20° C.) in the dark maintain about 96% of the initial SNAP species, compared to 89% for films stored at room temperature and 82% for films stored at 37° C. (see FIG. 8). The results shown in FIG. 8 illustrate the enhanced stability of SNAP during storage. Tertiary RSNOs, such as SNAP, have greater stability than primary RSNOs due to steric hindrance surrounding the sulfur atom. The increased thermal stability of SNAP in combination with the stabilization effect of the E2As polymer provides excellent storage stability of the SNAP/E2As material.

Stability of RSNOs has been studied for viscous polymer matrices containing such NO donors, including poly(ethylene glycol), PLURONIC® F127 hydrogel, poly(vinyl alcohol) and poly(vinyl pyrroloidone). RSNOs decompose according to the following sequence of reactions:

RSNO→RS●+NO● (1)

RS●+RSNO→RSSR+NO● (2)

With the overall reaction: 2RSNO→RSSR+2NO● (3).

The viscosity of the polymer matrix provides a cage effect on the bond cleavage and radical pair recombination. In addition, a viscous polymer matrix also limits the diffusion of the radical species, favoring geminate recombination to reform RSNO. Thus, the E2As polymer not only limits the diffusion of SNAP into the PBS, but it also appears to provide an additional stabilization effect to reduce the rate of SNAP decomposition.

An experiment was performed to test the storage stability of SNAP in the E2A matrix. FIG. 9 illustrates the results. In particular, FIG. 9 shows NO release behavior from 10 wt % SNAP in E2As dry film at 37° C. in the dark (n=1). The film was dried at 37° C. and then stored at 37° C. Approximately 5% of the total NO in the film was released during the first hours of storage, followed by very low levels of NO release. This corresponds with other storage/stability data disclosed herein (see FIG. 8), which shows that the SNAP loses its NO slowly during the 37° C., dry storage (losing only 10-15% of the SNAP after two months of storage in a dry state).

SNAP/E2As Coated ECC Loops and Effects on Rabbit Hemodynamics for Example 1

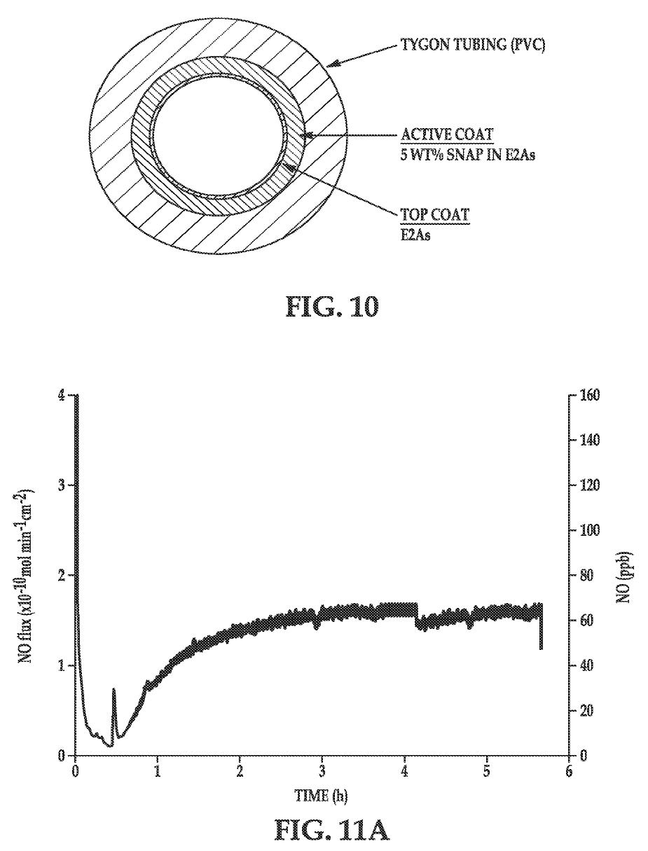

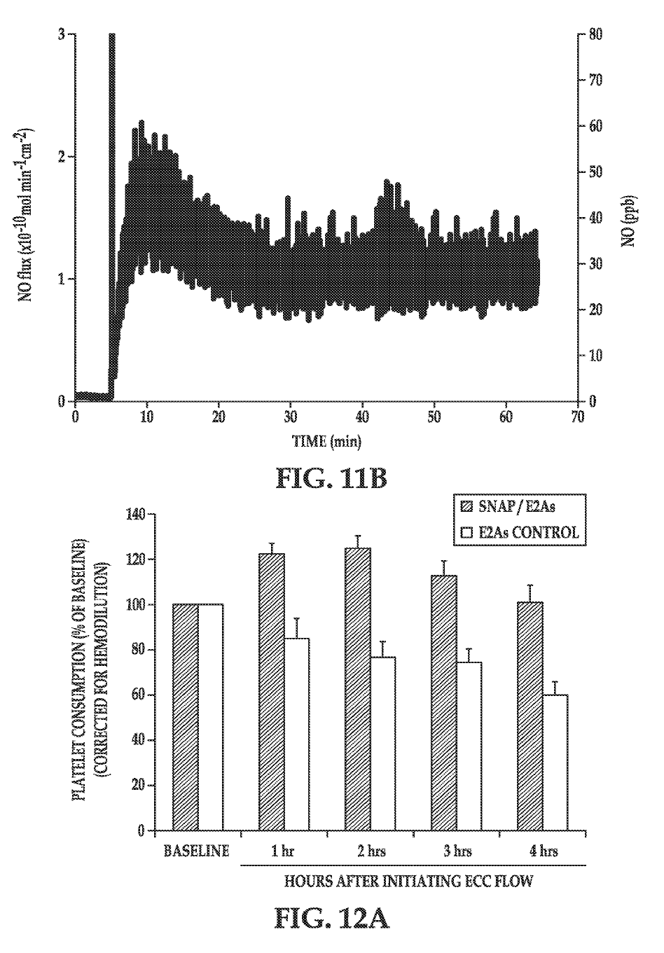

The active ECC loops coated with 5 wt % SNAP in E2As and a top coat of E2As (a schematic cross-section of which is shown in FIG. 10) and control loops coated with E2As only were prepared. 5 wt % SNAP was used in these tests due, in part, to the short duration of the ECC experiment. As described above, the SNAP/E2As coating has an initial burst of SNAP diffusing into solution during the first day of soaking. To reduce the effects of this burst during the short-term ECC experiments, all loops were first soaked overnight in saline, and the soaking solution was discarded prior to the ECC experiments. Nitric oxide released from samples of the coated ECC loops were measured with the NOA for NO release before blood exposure (after overnight soaking in saline). The NO release of the SNAP/E2As coated loops maintains an average flux of about 2× 10⁻¹⁰ mol cm⁻² min⁻¹ for 4 hours (at 37° C. with ambient light) (see FIG. 11A). After 4 hours of exposure to flowing blood, the ECC loops still exhibit a NO flux of at least 1.5×10⁻¹⁰ mol cm⁻² for at an additional 1 hour period (see FIG. 11B).

The hemodynamic effects of the SNAP/E2As coated ECC circuits were also monitored over the 4 hours of blood exposure in the rabbit ECC model. The mean arterial pressure (MAP) dropped significantly for both SNAP/E2As and control loops within the first hour, dropping to 35±2 mmHg and 46±2 mmHg, respectively. The MAP was maintained at these levels for the 4 hours by continuous IV fluid maintenance. The ECC blood flow dropped and remained at 64±5 mL/min for SNAP/E2As ECC, but maintained at baseline levels over the 4 hours (76±6 mL/min) for controls. The MAP drop and slower blood flow for the SNAP/E2As circuits is likely due to the vasodilatory effects of SNAP diffusing from the coating into the blood. The heart rate was maintained over the 4 hours and no significant difference was noted between the SNAP/E2As and control ECC loops, averaging 205±2 beats/min. The activated clotting time increased over the 4 hour period for both SNAP/E2As and control circuits, likely due to the increase in intravascular fluids (the hemodilution effect). Similar effects on MAP and flow rate have been observed with SNAP infusion.

Effects of SNAP/E2As Coatings on Rabbit Platelet Function and Thrombus Formation for Example 1

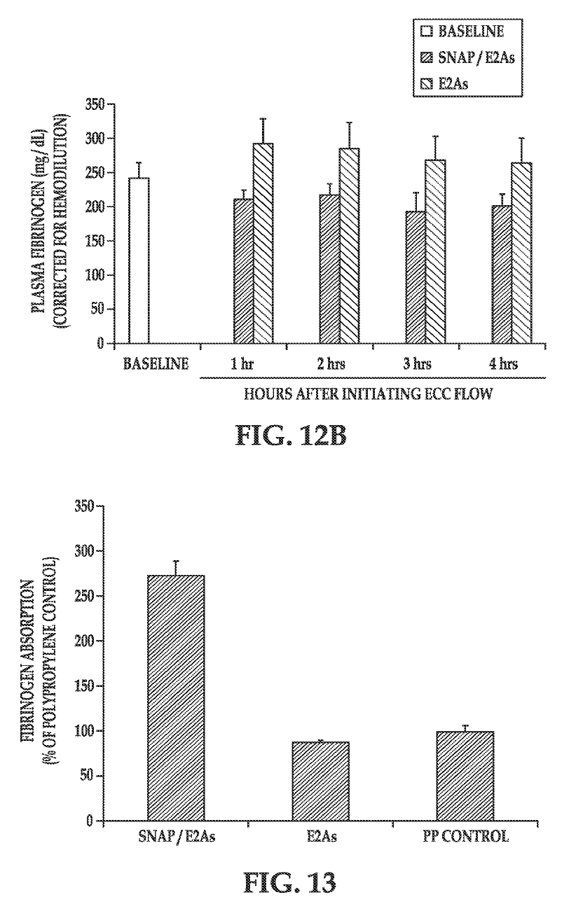

Platelet activation and function throughout the 4 hour ECC was assessed by recording the platelet count (e.g., consumption, see FIG. 12A) and plasma fibrinogen levels (see FIG. 12B), which were both corrected for hemodilution due to the added IV fluids, as well as % platelet aggregation. The baseline platelet counts (×10⁸ platelets/mL) were 3.5±0.6 and 4.8±0.5 for the 5 wt % SNAP/E2As and E2As control circuits, respectively. For the SNAP/E2As circuits, the platelet count initially rose slightly and was maintained at 100±7% of baseline levels at the end of 4 hours on ECC. The platelet count for control circuits exhibited a time-dependent loss in platelets, dropping to 60±6% of baseline after 4 hours. The percent of platelet functional aggregation was determined by ex vivo collagen stimulation of PRP and measured by optical turbidity. The platelets from blood taken from circulation through the SNAP/E2As and control circuits showed similar response to collagen-stimulated platelet aggregation during the 4 hour blood exposure, both maintaining 56±12% (with baseline values at 68±6%).

As shown in FIG. 12B, the plasma fibrinogen levels were maintained at baseline levels for the control circuits. For the 5 wt % SNAP/E2As circuits, the plasma fibrinogen levels during the first hour of ECC dropped to 83% of baseline levels and remained at that level for the 4 hour ECC. This decrease in plasma fibrinogen levels can be attributed to fibrinogen binding to the surfaces, as shown by the in vitro fibrinogen assay results (see FIG. 13). Surprisingly, even with the enhanced adsorption of fibrinogen on the SNAP/E2As coatings, these materials still exhibited significantly less platelet loss than controls, suggesting that the levels of NO produced overcome the enhanced fibrinogen adsorption that would normally enhance activation of platelets.

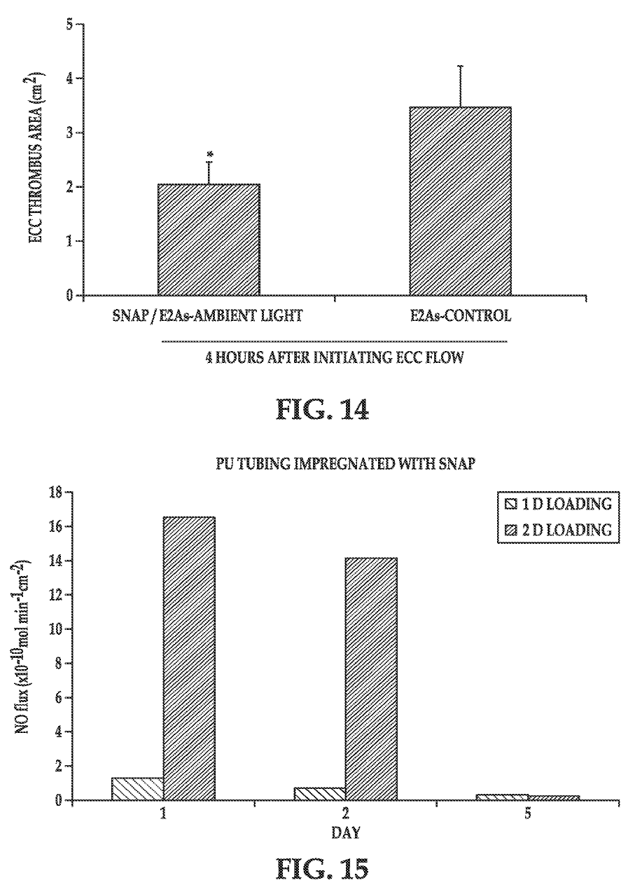

To determine the differential formation of thrombus in the thrombogenicity chamber of the ECC circuit (i.e., the ⅜ inch ID Tygon® tubing, 8 cm in length within the ECC loop), 2-dimensional (2D) image analysis was performed after 4 hours of blood exposure. The thrombus area was analyzed by using Image J software and represents the 2D arca of thrombus formation (pixels/cm²) in each thrombogcnicity chamber. The thrombus area was quantitated and data are shown in FIG. 14. The thrombus area is significantly reduced for the SNAP/E2As circuits when compared to controls, although the E2As controls also had relatively low thrombus area, likely resulting from the enhanced intrinsic biocompatibility of the E2As polymer.

One of the effects of the SNAP/E2As coating is the hypotension caused by the diffusion of SNAP into the blood stream. The co-administration of intravenous fluids counteracts this, but may in some instances pose difficulties in a clinical situation. Applications of SNAP have been reported to cause hypotension, hyperglycemia and impaired insulin secretion, and decreased cell viability. However, when used as catheters for coatings for small implantable devices, the surface area to volume (of blood) ratios will be quite small, and thus the amounts of SNAP lost to the blood will generally not be a significant issue. Furthermore, endogenous thiols and superoxide dismutase will reduce many of the adverse effects. The parent thiol, N-Acetyl-DL-penicillamine (NAP), however, has been used clinically to treat mercury poisoning and cystinuria with minimal side effects. Although the SNAP/E2As coatings disclosed herein do exhibit a hypotension effect, the daily levels of SNAP delivered by the coating are well below the reported levels causing the other potential adverse side effects described above.

Impregnation Method for Making SNAP-Doped Polymers for Example 1

The present disclosure further includes a novel method for loading commercial SR or PU tubing with SNAP. Commercial SR and PU include medical grade tubing, including those available from US plastics, Cole Palmer, Professional Plastics, ICORally, and Thermedics, Inc. FIG. 15 shows an NO release profile of polyurethane (PU) tubing that was soaked in a SNAP/acetone solution for either 1 or 2 days. The tubing was then rinsed with acetone and dried prior to the NOA testing. The tubing was soaked in PBS at 37° C. for the NOA testing.

This impregnation approach enables the incorporation of the SNAP species within the walls of existing commercial catheters/tubings. This approach avoids problems that may arise when attempting to extrude SNAP into a polymer tubing under normal hot extrusion conditions due to the thermal instability of NO donors (e.g., SNAP and related species) at high temperatures. While acetone was used in the impregnation approach described in Example 1, other solvents (or mixtures of solvents) may be used include ethyl acetate, cyclohexane, isopropanol, methanol, butanone, tetrahydrofuran (THF), etc. According to one embodiment of the novel method, a mixture of solvents comprising THF is preferable for use with silicon-containing polymers because THF swells the catheter without compromising its structural integrity while dissolving SNAP.

SNAP-Doped Polymers for Catheter Tubing Applications for Example 1

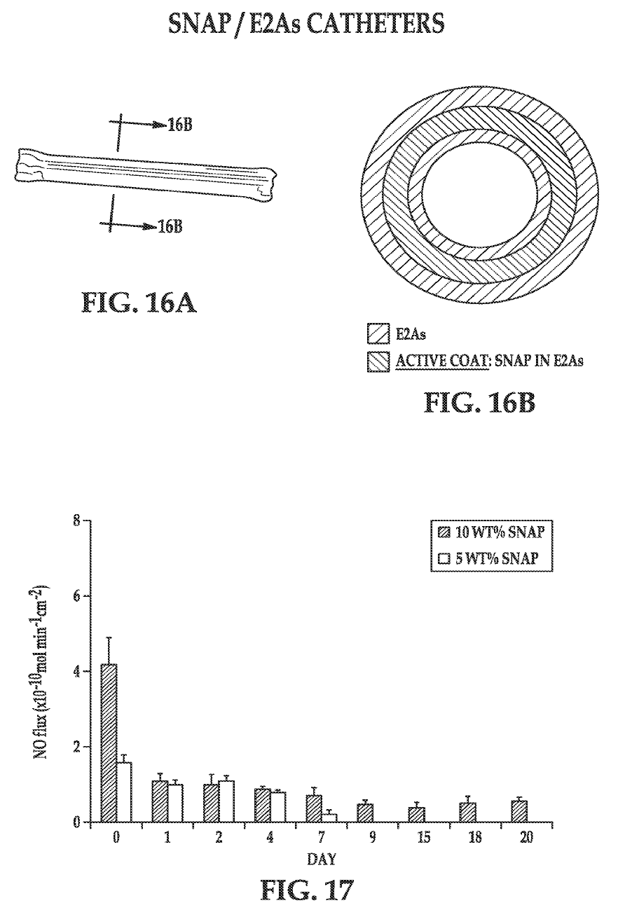

FIG. 16A is a schematic illustration of an E2As catheter tubing prepared with 5 wt % or 10 wt % SNAP/E2As according to an example of the present disclosure followed by the application of a top coat of E2As. FIG. 16B is a cross-sec tion of the catheter tubing, illustrating the various layers. In general, the trilayer catheters were prepared by dip-coating 5 base coats of an E2As solution, 25 coats of the respective active solutions, and 5 top coats of the E2As solution. The SNAP/E2As catheters were kept in phosphate buffered saline (PBS) in the dark at 37° C.

FIG. 17 shows 20 day NO release of the SNAP/E2As catheters at 37° C. in the dark in the phosphate buffered saline (PBS). The results in FIG. 17 illustrate that the 10 wt % SNAP/E2As catheters were able to release NO for up to 20 days and that the 5 wt % SNAP/E2As catheters were able to release NO for up to 7 days at the specified conditions.

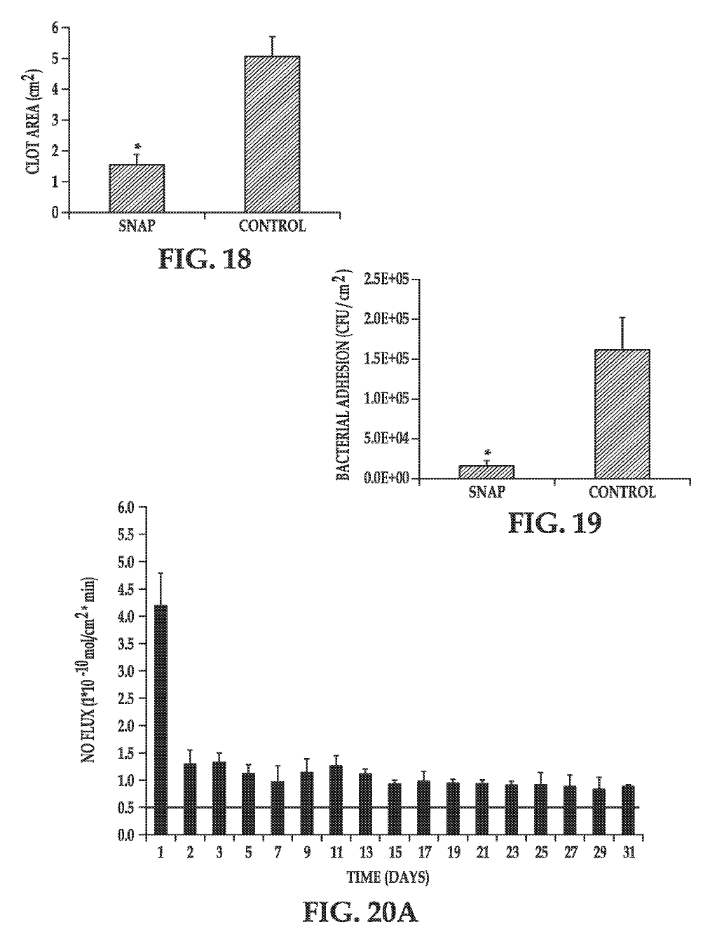

E2As control catheters and the 10 wt % SNAP/E2As catheters were implanted in sheep veins for 7 days. After explanation, the SNAP/E2As catheters were found to have significantly less thrombus (FIG. 18) and a 90% reduction of bacterial adhesion (FIG. 19) than the E2As control catheters. Together FIGS. 17 through 19 demonstrate the potential application of the SNAP-doped polymers in the catheter configuration.

Materials for Example 1

N-Acetyl-D,L-penicillamine (NAP), sodium chloride, potassium chloride, sodium phosphate dibasic, potassium phosphate monobasic, ethylenediaminetetraacetic acid (EDTA), tetrahydrofuran (THF), sulfuric acid and N,N-dimethylacetamide (DMAc) were purchased from Sigma-Aldrich (St. Louis, Mo.). Methanol, hydrochloric acid and sulfuric acid were obtained from Fisher Scientific (Pittsburgh, Pa). TECOPHILIC™ SP-60D-60 and TECOFLEX™ Sg-80A were products of Lubrizol Advanced Materials Inc. (Cleveland, Ohio). Dow Corning RTV 3140 Silicone Rubber (SR) was purchased from Ellsworth Adhesives (Germantown, Wis.). CARBOSIL® 20 90A was from the Polymer Technology Group (Berkeley, Calif.). ELAST-EON™ E2As was obtained from AorTech International, PLC (Scoresby, Victoria, Australia). Human plasma fibrinogen containing >90% clottable proteins was a product of Calbiochem (La Jolla, Calif.), and fluorescein-labeled goat IgG (polyclonal) against denatured human fibrinogen was purchased from MP Biomedicals, LLC (Solon, Ohio). Black, polypropylene 96-well microtiter plates used for fluorescence measurements were obtained from Nalge Nunc International (Rochester, N.Y.). All aqueous solutions were prepared with 18.2 MΩ) deionized water using a Milli-Q filter (Millipore Corp., Billerica, Mass.). Phosphate buffered saline (PBS), pH 7.4, containing 138 mM NaCI, 2.7 mM KCl, 10 mM sodium phosphate, 100 μM EDTA was used for all in vitro experiments.

Synthesis of SNAP for Example 1

SNAP was synthesized using a modified version of a previously reported method (I. Chipinda, R. H. Simoyi, Journal of Physical Chemistry B 2006, 110, 5052). Briefly, an equimolar ratio of NAP and sodium nitrite was added to a 1:1 mixture of water and methanol containing 2 M HCl and 2 M H₂SO₄. After 30 minutes of stirring, the reaction vessel was cooled in an ice bath to precipitate the green SNAP crystals. The crystals were collected by filtration, washed with water, and allowed to air dry. The reaction and crystals were protected from light at all times.

Preparation of SNAP-Doped Films for Example 1

The polymer films containing 5 wt % and 10 wt % SNAP were prepared by solvent evaporation. For the 10 wt % SNAP films, the casting solutions were prepared by dissolving 180 mg of the respective polymer in THF. The polyurethanes (SP-60D-60, SG-80A, CARBOSIL® and ELAST-EON™ E2As) were dissolved in 3 mL THF, and SR was dissolved in 1 mL THF. SNAP (20 mg) was then added to the polymer solution, and the mixture was stirred for 10 minutes. The 5 wt % SNAP films were prepared similarly with SNAP (10 mg) and polymer (190 mg). The film solution was cast in a TEFLON® ring (d=2.5 cm) on a Teflon® plate and dried overnight under ambient conditions. Small disks (d=0.7 cm) were cut from the parent films and were dip coated 2 times with a topcoat solution (200 mg of the respective polymer (no SNAP added) in 4 mL THF) and dried overnight. As such, the topcoat for each sample was made of the same polymer as the parent film. The weight of each small disk was recorded prior to topcoating. All films and film solutions were protected from light. The final films had a SNAP-doped layer that was about150 μm thick and a top coat that was about 50 μm thick.

Preparation of SNAP/E2As Coated ECC Loops for Example 1

The ECC configuration employed in the in vivo rabbit study consisted of 16-gauge and 14-gauge IV polyurethane angiocatheters (Kendall Monoject Tyco Healthcare, Mansfield, MA), two 16 cm in length ¼ inch inner diameter (ID) Tygon® tubing, and an 8 cm length of ⅜ inch ID Tygon® tubing that created a thrombogenicity chamber where thrombus could form more easily due to more turbulent blood flow.

As previously mentioned, due to the short duration of the ECC experiments (4 hours), the NO release ECC loops were coated with only 5 wt % SNAP in E2As. The SNAP/E2As solution was prepared by dissolving SNAP (125 mg) and E2As (2375 mg) in THF (15 mL). The E2As control solution consisted of E2As in (2500 mg in 15 mL). SNAP/E2As loops were first coated with 2 layers of the SNAP/E2As solution, followed by 1 coat of the E2As control solution. E2As control loops were coated with 2 coats of the E2As control solution. ECC loops were allowed to air dry for 1 hour in the dark between each coat. The completely coated ECC was welded together using THF, starting at the left carotid artery side, with the 16-gauge angiocatheter, one 15 cm length ¼ inch ID tubing, the 8 cm length thrombogenicity chamber, the second 15 cm length ¼ inch ID tubing and finally the 14-gauge angiocatheter. The angiocatheters were interfaced with tubing using two luer-lock PVC connectors. The assembled ECC loops were dried under vacuum while protected from light for at least 48 hours. Prior to the ECC experiment, the loops were filled with saline solution for overnight soaking, and this solution was discarded immediately before the rabbit experiment.

In Vitro Characterization of SNAP-Doped Films for Example 1

UV-Vis Spectra

All UV-Vis spectra were recorded in the wavelength range of 200 nm-700 nm using a UV-Vis spectrophotometer (Lambda 35, Perkin-Elmer, Mass.) at room temperature. The presence of the S-NO group of SNAP provides characteristic absorbance maxima at 340 nm and 590 nm, corresponding to the it π→π* and nN→π* electronic transitions.

Diffusion of SNAP from SNAP-Doped Polymer Films Immersed in PBS

Top coated films were placed in individual vials soaked in 10 mM PBS, pH 7.4, containing 100 μM EDTA to minimize any trace metal ion catalyzed decomposition of SNAP. Films were incubated in the dark at room temperature (22° C.) or 37° C. At various time points, the UV-Vis spectra of a 1 mL aliquot of the PBS was taken for rapid determination of the SNAP concentration. The aliquots were protected from light and were immediately returned to the sample vials for the duration of the experiment. The films were placed in fresh PBS buffer daily. The molar absorption coefficient for SNAP in PBS at 340 nm was determined to be: ε_{SNAP}=1024 M⁻¹ cm⁻¹.

Cumulative NO Release from SNAP/E2As Films

After the 10 wt % SNAP in E2As films were prepared, the UV-Vis spectra were recorded of individual films dissolved in DMAc to determine the initial concentration of SNAP within the films (nmol SNAP/mg film). Equivalent films were then placed in individual vials containing 3 mL PBS (pH 7.4) containing 100 μM EDTA. Films were incubated under various conditions: RT under ambient light, 37° C. under ambient light, 37° C. in dark, and 37° C. under a 100W floodlight. The fluorescent lights in the laboratory are referred to as ambient light. Films were placed in fresh PBS daily. At various time points, the films were dissolved in DMAc for rapid determination of the SNAP present in the film. The amount of NO released was determined indirectly from the amount of SNAP decomposed at various time points. The cumulative NO released over time ([NO]ₜ) was calculated by the difference between the initial amount of SNAP in the film ([SNAP]₀) and the amount of SNAP at time t ([SNAP]ₜ): [NO]ₜ=[SNAP]₀-[SNAP]ₜ (where concentrations are in nmol/mg film). This calculation was based on the fact that the decay of the 340 nm absorption band of SNAP is directly associated with the hemolytic cleavage of the S-NO bond and concomitant NO release. The molar absorption coefficient for SNAP in DMAc at 340 nm was determined to be: ε_{SNAP}=1025 M⁻¹ cm⁻¹.

NO Release Measurements

Nitric oxide released from the films was measured using a Sievers chemiluminescence Nitric Oxide Analyzer (NOA) 280 (Boulder, Colo.). Films were placed in the sample vessel immersed in PBS (pH 7.4) containing 100 μM EDTA. Nitric oxide was continuously purged from the buffer and swept from the headspace using an N₂ sweep gas and bubbler into the chemiluminescence detection chamber. Clear glass sample vessels were used for the ambient light and photoinitiated NO release experiments. A 100W halogen floodlight (GE model 17986) was placed about 60 cm from the sample cell for the photolysis experiments. Films were incubated in the PBS under the same conditions as the NOA measurements (ambient light or 100W floodlight irradiation at 37° C.).

SNAP/E2As Stability Study

SNAP/E2As films (consisting of 10 wt % SNAP) were placed under the following conditions in vials with desiccant: room temperature with ambient light, room temperature in dark, 37° C. in dark, and in the freezer (−20° C.) in dark. At various time points over a 4 month period, films were dissolved in DMAc, and the UV-Vis spectra was recorded to determine the % SNAP remaining in the film, as compared to the initial 10 wt % SNAP.

In Vitro Fibrinogen Adsorption Assay

The in vitro fibrinogen adsorption immunofluorescence assay was performed in a 96-well format. The SNAP/E2As and E2As control polymer solutions used to prepare the ECC circuits were also employed to coat microwells of the 96-well microliter plates and were dried under the same conditions as the ECC loops. Briefly, human fibrinogen was diluted to 3 mg/mL with Dulbecco's phosphate-buffered saline (dPBS) without CaCl₂ and MgCl₂ (Gibco Invitrogen, Grand Island, N.Y.), equivalent to the human plasma concentration, and then used for adsorption experiments. One hundred μL of this solution were added to each well and the coated wells were incubated with this solution for 1.5 hours at 37° C. This was followed by eight washing steps using wash buffer (100 μL) for each wash, which consisted of a 10-fold dilution of the AbD Serotec Block ACE buffer (Raleigh, N.C.) containing 0.05% Tween 20 (Calbiochem La Jolla, Calif.). To block nonspecific antibody binding, coated wells were incubated with 100 μL of blocking buffer (4-fold dilution of Serotec Block ACE buffer) for 30 minutes at 37° C. After rinsing 3 times with wash buffer (100 μL per well), a background fluorescence measurement of the plates was performed at 485 nm (excitation) and 528 nm (emission) on a Synergy 2 fluorescence microplatc reader (Biotek Winooski, Vt.). To detect the adsorbed fibrinogen, fluorescein-labeled goat anti-human fibrinogen antibody was diluted (1:10) in a 10-fold dilution of the Serotec Block ACE buffer and 100 μL of this final solution was added to each well. The antibody was allowed to bind to the surface-adsorbed fibrinogen for 1.5 hours at 37° C. Human fibrinogen adsorption to non-coated polypropylene was used as an internal control to normalize the fluorescence signals within different plates. All measurements were conducted in triplicate.

Rabbit ECC Thrombogenicity Experiments for Example 1

All animal handling and surgical procedures employed were approved by the University Committee on the Use and Care of Animals in accordance with university and federal regulations. A total of 8 New Zealand white rabbits (Covance, Battle Creek, Mich.) were used in this study. All rabbits (2.5 kg-3.5 kg) were initially anesthetized with intramuscular injections of 5 mg/kg xylazine injectable (AnaSed® Lloyd Laboratories Shenandoah, Iowa) and 30 mg/kg ketamine hydrochloride (Hospira, Inc., Lake Forest, Ill.). Maintenance anesthesia was administered via isoflurane gas inhalation at a rate of 1.5%-3% via mechanical ventilation which was done via a tracheotomy and using an A.D.S. 2000 Ventilator (Engler Engineering Corp. Hialeah, Fla.). Peek inspiratory pressure was set to 15 cm of H₂O, and the ventilator flow rate set to 8 L/min. In order to aid in maintenance of blood pressure stability, IV fluids of Lactated Ringer's were given at a rate of 10 mL/kg/h For monitoring blood pressure and collecting blood samples, the rabbits' right carotid artery were cannulated using a 16-gauge IV angiocatheter (Jelco®, Johnson & Johnson, Cincinnati, Ohio). Blood pressure and derived heart rate were monitored with a Series 7000 Monitor (Marquette Electronics Milwaukee, Wis.). Body temperature was monitored with a rectal probe and maintained at 37° C. using a water-jacketed heating blanket. Prior to placement of the arteriovenous (AV) custom-built extracorporeal circuit (ECC), the rabbit left carotid artery and right external jugular vein were isolated and baseline hemodynamics as well as arterial blood pH, pCO₂, pO₂, total hemoglobin and methemoglobin were measured using an ABL 825 blood-gas analyzer and an OSM3 Hemoximeter (Radiometer Copenhagen, DK). In addition, baseline blood samples were collected for platelet and total white blood cell (WBC) counts which were measured on a Coulter Counter Z1 (Coulter Electronics Hialeah, Fla.). Plasma fibrinogen levels were determined using a Dade Behring BCS Coagulation Analyzer (Siemens, Deerfield, Ill.), activated clotting times (ACT) were monitored using a Hemochron Blood Coagulation System Model 801 (International Technidyne Corp., Edison, N.J.), and platelet function was assessed using a Chrono-Log optical aggregometer model 490 (Havertown, Pa.).

After baseline blood measurements, the AV custom-built ECC was placed into position by cannulating the left carotid artery for ECC inflow and the right external jugular vein for ECC outflow. The flow through the ECC was initiated by unclamping the arterial and venous sides of ECC, and blood flow in circuit was monitored with an ultrasonic flow probe and flow meter (Transonic HT207, Ithaca, N.Y.). Animals were not systemically anticoagulated during the experiments.

After 4 hours on ECC, the circuits were clamped, removed from animal, rinsed with 60 mL of saline and drained. Any residual thrombus in the larger tubing of ECC (i.e., thrombogenicity chamber) was photographed, and the degree of thrombus was quantitated using Image J imaging software from National Institutes of Health (Bethesda, Md.). Prior to euthanasia, all animals were given a dose of 400 U/kg sodium heparin to prevent necrotic thrombosis. The animals were euthanized using a dose of Fatal Plus (130 mg/kg sodium pentobarbital) (Vortech Pharmaceuticals, Dearborn, Mich.). All animals underwent gross necropsy after being euthanized, including examination of the lungs, heart, liver and spleen for any signs of thromboembolic events.

Blood Sampling for Example 1

Rabbit whole blood samples were collected in non-anticoagulated 1 cc syringes for ACT, and in 3.2% sodium citrate vacutainers (Becton, Dickinson, Franklin Lakes, N.J.) with 3 cc volumes for cell counts and aggregometry, and 1 cc syringes containing 40 U/mL of sodium heparin (APP Pharmaceuticals, LLC, Schaumburg, Ill.) for blood-gas analysis. Following the initiation of ECC blood flow, blood samples were collected every hour for 4 hours for these in vitro measurements. Samples were used within 2 hours of collection to avoid any activation of platelets, monocytes or plasma fibrinogen.

Platelet Aggregometry for Example 1

Rabbit platelet aggregation was assayed based on the Born's turbidimetric method using a Chrono-Log optical aggregometer. Briefly, citrated blood (1:10 blood to 3.2% sodium citrate solution) was collected (6 mL), and platelet-rich plasma (PRP) was obtained by centrifugation at 110×g for 15 minutes. Platelet-poor plasma (PPP) was obtained by another centrifugation of the PRP-removed blood sample at 2730×g for 15 minutes and was used as the blank for aggregation.

PRP was incubated for 10 minutes at 37° C. and then 25 μg/mL collagen (Chrono-PAR #385 Havertown, Pa.) was added. The percentage of aggregation was determined 3 minutes after the addition of collagen using Chrono-Log Aggrolink software.

Preparation of SNAP-Doped E2As and E2As Control Catheters for Example 1

Catheters were prepared by dip-coating polymer solutions on 18 cm long stainless steel mandrels of 2 mm diameter (purchased from McMaster Carr). For the E2As control catheters, the polymer solution consisted of E2As dissolved in THF (150 mg/mL). Thirty-five coats of the E2As solution was applied on the mandrel by dip-coating at an interval of 2 min between each coat. For the SNAP/E2As catheters, two different solutions, namely a top/base coat solution and an active solution, were prepared to make the trilayer catheters (see FIG. 16). The top/base coat solution consisted of E2As dissolved in THF (150mg/mL). The active solution was made up of 10 wt % SNAP and 90 wt % E2As was dissolved in THF with overall concentration of 150 mg/mL. Trilayer catheters were prepared by dip-coating 5 base coats of E2As solution, 25 coats of active solution, and 5 top coats of E2As solution. Catheters used for sheep studies were 15 cm long.

Long-Term (7 day) Implantation of Catheters in Sheep for Example 1

Sheep Catheter Implantation

All animals received care compliant with the “Principles of Laboratory Animal Care” formulated by the National Society for Medical Research and the “Guideline for the Care of Use of Laboratory Animals” prepared by the National Academy of Sciences and published by the N1H. This study was approved by the University of Michigan Committee on Use and Care of Animals.

Five adult sheep were utilized in the large animal model. All experiments were performed under sterile conditions. The first 3 sheep procedures were performed using 1% Lidocaine subcutaneously for local anesthetic. Small (1 cm to 2 cm) vertical and transverse incisions were created over the right and left jugular vein. Cannulas were then placed using a modified Seldinger technique with one cannula either control (E2As) or experimental (SNAP/E2As) placed in either the right or left jugular vein. The skin was then re-approximated using skin staples.

The final 2 sheep experiments were performed under general anesthetic. Propofol (1 mg/kg) was used for induction followed by Isoflurane (0.1-4%) anesthetic for maintenance. A small 2-3 cm incision was created overlying the jugular vein. The right and left jugular veins were then isolated and either control (E2As) or experimental (SNAP/E2As) cannulas were placed under direct visualization. Sheep were then recovered and returned to animal housing.

Animals remained in animal housing throughout the remainder of the experiment. Catheters were tested on a daily basis for patency. Cannulas were initially attempted to be aspirated and then flushed with 15 mL of saline. If aspiration was initially difficult, 15 mL of normal saline was attempted to be infused, and the process and aspiration and flushing were again tested. Patency data was recorded every 24 hour for 7 days.

Necropsy was performed on day 7. Sheep were anesthetized using the same anesthetic protocol described above. The right and left jugular veins were dissected along their length and isolated. The sheep were heparinized using approximately 100-150 IU/kg bolus dose and activated clotting time of >200 seconds was confirmed. The jugular veins were then ligated and opened longitudinally. Catheters were removed and placed in sterile saline for further analysis.

Catheter Evaluation

After explanting, the catheters were rinsed in PBS. Pictures were taken of the exterior of the whole catheter and the interior of a 1 cm piece cut longitudinally using a Nikon L24 digital camera. The degree of thrombus was quantitated using Image J imaging software from NIH. To quantitate the viable bacteria, a 1 cm piece was cut longitudinally and was homogenized in 1 mL PBS buffer. The optimal homogenizing speed was found using a separate experiment where different homogenizing speeds and times were compared. The resulting homogenate was serially diluted in sterile PBS. Triplicate aliquots of each dilution (10 μL) were plated on agar plates. The agar plates were incubated at 37° C. for 24 hours, followed by calculation of colony forming units per catheter surface area (CFU/cm²).

Statistical Analysis for Example 1

Throughout this disclosure, data are expressed as mean±SEM (standard error of the mean). Comparison between the various SNAP/E2As and E2As control polymer groups were analyzed by a comparison of means using student's t-test. Values of p<0.05 were considered statistically significant for all tests.

Conclusions for Example 1

Examples of the present disclosure have shown that hydrophobic polyurethanes, e.g., siloxane-based polyurethane elastomers (one example of which is the ELAST-EON™ E2As polymer) are excellent matrices to act as a reservoir for SNAP, and the resulting films can be used for the controlled release of NO and SNAP. SNAP slowly diffuses from the polymer film, and NO release from the film/coating can be initiated by light and/or thermal decomposition when blood flows through an ECC loop. A stability study demonstrates that SNAP is quite stable within the E2As matrix, even during storage for 4 months at 37° C. While the E2As polymer has excellent innate biocompatible properties on its own, incorporating SNAP into the E2As polymer matrix provides controlled delivery of NO/SNAP to further improve polymer hemocompatibility. The SNAP/E2As coated ECC loops significantly preserved platelet count and function during 4 hours of ECC blood flow, while also reducing the clot area when compared to corresponding E2As coated control loops. In addition, the NO released from SNAP/E2As catheters was able to significantly reduce thrombus and bacterial adhesion during 7 day implantation in sheep, thereby improving catheter patency. Incorporating SNAP within ELAST-EON™ E2As polymer films/coatings provides a simple way to locally deliver NO/SNAP, and has potential for improving the hemocompatibility of a wide variety of blood-contacting medical devices.

In summary, examples as disclosed herein include novel nitric oxide (NO) releasing coatings including siloxane-based polyurethane elastomers doped with S-nitroso-N-acetylpenicillamine (SNAP) to prevent thrombus formation in, e.g., extracorporeal circulation (ECC) circuits and catheter tubing. In addition, the NO release from these formulations is likely to serve as a very effective bacterial agent.

### EXAMPLE 2

In this example, the results of embodiments of the novel impregnation method were further studied. SNAP-impregnated silicone Foley catheters were generated via a solvent swelling method. As will be discussed in more detail herein, the catheters of Example 2 generated NO surface-fluxes >0.7 ×10⁻¹⁰ mol min⁻¹ cm⁻² for over one month under physiological conditions, with minimal SNAP leaching; and prevented biofilm formation over 3, 7, and 14 day periods by microbial species commonly causing CAUTIs. In Example 2, it is also shown that segments of SNAP impregnated Foley catheter tubings reduced the colony-forming units (CFU) of gram-positive S. epidermidis biofilms grown over 14 days by almost 4 log units. Further, the SNAP-impregnated silicone Foley catheters of Example 2 abated cell viability of biofilms by 6 log units formed over 14 days by gram-negative Proteus mirabilis, the principle bacterium in causing urinary catheter encrustation infections. Finally, the toxicity assessment data in Example 2 demonstrates that SNAP-impregnated silicone Foley catheters are fully biocompatible (as extracts of the catheter tubings scored 0 on a 3-point grading scale using an accepted mouse fibroblast cell-line toxicity model).

Materials for Example 2

N-Acetyl-D,L-penicillamine (NAP), sodium chloride, potassium chloride, sodium phosphate dibasic, potassium phosphate monobasic, ethylenediaminetetraacetic acid (EDTA), and tetrahydrofuran (THF) were purchased from Sigma Aldrich (St. Louis, Mo.), and used as received. Methanol (MeOH), hydrochloric acid (HCl) and sulfuric acid (H₂SO₄) were obtained from Fisher Scientific (Pittsburgh, Pa.). Luria Bertani (LB) broth and LB agar were also obtained from Fisher Scientific Inc. Aqueous solutions were prepared with deionized water using a Milli-Q filter (18MΩ cm⁻¹; Millipore Corp., Billerica, Mass.). Phosphate buffered saline (PBS), pH 7.4, containing 10 mM sodium phosphate, 138 mM NaCl, 2.7 mM KCl, and 100 μ EDTA was used for all in vitro experiments of Example 2.

Silicone 2-way Foley Balloon Catheters, size 18 Fr (outer diameter (o.d.)=0.59 cm, inner diameter (i.d.)=0.30 cm), were purchased from Fortune Medical Instrument Corp (New Taipei City, TAIWAN). S. epidermidis ATCC 14990 and P. mirabilis ATCC 29906 were obtained from the American Type Culture Collection (ATCC) (Manassas, Va.).

Synthesis of SNAP for Example 2

SNAP was synthesized using the method described in Example 1, except that an equimolar ratio of NAP and sodium nitrite was added to a 3:1 mixture by volume of methanol and water containing 2 M HCl and 2 M H₂SO₄.

Preparation of SNAP Impregnated Silicone Foley Catheters for Example 2

SNAP was first dissolved in THF at a concentration 125 mg/mL of solvent, for 15 minutes. Existing FDA approved silicone Foley catheter tubing was cut either into 1 cm long sections (for NO release measurements, SNAP % loading and leaching assessments) or into 2 cm long sections (for biofilm studies). All of the segments were completely immersed into SNAP-containing THF solutions, within glass vials, for 24 hours in the dark. After this swelling/impregnation period, the catheter sections were dried in dark for 72 hours within a fume hood to remove any residual solvent. Control Foley catheter segments (designated for the biofilm experiments) were swollen in a vial containing only THF for 24 hours, followed by a 72 hour drying process within the fume hood.

NO Release Measurements for Example 2

Nitric oxide release from the surfaces of 1 cm SNAP impregnated Foley catheter segments (total surface area=3.19 cm²) was measured using a Sievers chemiluminescence Nitric Oxide Analyzer (NOA) 280i (Boulder, Colo.). The catheter segments were placed in an amber glass vessel (placed in a water bath at 37° C.) containing 4 mL of PBS (pH 7.4) with 100 μM EDTA. Nitric oxide released from the catheter segment was continuously purged from the buffer and swept from the headspace using an N₂ sweep gas and bubbler into the chemiluminescence detection chamber. When not being tested with the NOA, the SNAP impregnated catheters were incubated in 10 mM PBS (pH 7.4) with 100 μM EDTA at 37° C., avoiding exposure to light. All experiments were conducted in triplicate.

SNAP Loading Efficiency for Example 2

Since the reaction of 2 RSNO→RSSR+2 NO is catalyzed by light in the visible region of the spectrum, the total loading of SNAP into the 1 cm catheter segments was determined by the total amount of NO (in moles) released over time in the presence of a high intensity white light source (until no additional NO could be detected above baseline by the NOA). By integrating the signal from the NOA over time, the total amount of SNAP impregnated into each catheter segment was calculated. The wt % of SNAP within the catheter material was quantitated using the initial weight of the catheter segment.

NO release was measured via the NOA, by immersing the catheter tubing sample in a clear glass vessel (kept inside a water bath at 37° C.) containing 50 mM CuCl₂ and 10 mM cysteine. The added cysteine catalyzed the reduction of Cu²⁺ into Cu⁻, which then promoted the SNAP decomposition and the release of NO. A 100W halogen floodlight (GE model 17986) was placed 20 cm away from the sample, and was used to photo-initiate the NO release from the SNAP impregnated catheter pieces. All experiments were conducted in triplicate.

SNAP Leaching from Surface of SNAP Impregnated Foley Catheters for Example 2

1 cm long catheter segments were completely immersed into 1 mL of 10 mM PBS (with 100 μM EDTA) inside individual amber glass vials and stored at 37° C. for the entire duration of the experiment. At every time point, each of the soaking solutions, where catheter segments had been stored, was tested by UV-Vis to detect the SNAP characteristic UV-vis absorbance band maximum at 340 nm. Using the Beer-Lambert law (A=cεl), the moles of SNAP leached out at every time point was calculated. Since the wt % of total SNAP loading was determined from total NO release (see SNAP loading efficiency for Example 2), the % of leached SNAP can be quantitated. All UV-Vis spectra were recorded in the wavelength range of 250-650 nm using a UV-Vis spectrophotometer (Lambda 35, PerkineElmer, Mass.). The presence of the S-NO group of SNAP provides characteristic absorbance maxima at 340 nm and 590 nm, corresponding to the π_{→}π* and n_{N+→}*π* electronic transitions. The molar absorption coefficient for SNAP in PBS at 340 nm was determined to be ε_{SNAP}=1075M⁻¹ cm⁻¹. PBS was used as the blank and all experiments were conducted in triplicate.

Biofilm Growth Conditions and Plate Counting

S. epidermidis Biofilm

S. epidermidis ATCC 14990 was tested as a bacterial strain to develop 3, 7, and 14 day-old biofilms on the surfaces of both control and SNAP impregnated silicone Foley catheter segments. A CDC biofilm reactor (Biosurface Technologies, Bozeman, Mont.) was used for the biofilm growth. The CDC biofilm reactor and its coupon holders were autoclaved before use. Both control and SNAP impregnated catheters segments were mounted on the coupon holders and the reactor was supplemented with 10% LB medium by a peristaltic pump with a continuous flow rate of 100 mL/h. Overnight cultures of S. epidermidis (grown under shaking conditions at 37° C.) were diluted by 1:100 and inoculated into the glass vessel of the CDC reactor aseptically. The liquid growth medium was circulated through the vessel and a shear force was generated by a magnetic stir bar rotated by a magnetic stir plate. The CDC biofilm reactor was placed inside an incubator and biofilms were grown at 37° C. After 3, 7 or 14 days of growth, the catheters were aseptically removed and each catheter was cut into two 1 cm segments.

One of the 1 cm segments was utilized for fluorescent imaging of live bacteria on the surface, while the other 1 cm segment was used for plate count experiments. Each 1 cm segment to be used for plate counting was placed into a centrifuge tube, containing 2 mL of 10 mM sterilized PBS (pH 7.2). The 1 cm segment was then homogenized for 30 seconds in order to disintegrate the biofilm clumps and form a homogeneous single cell suspension. Finally, to assess cell viability, samples were 10-fold serially diluted and plated onto LB agar plates. All experiments were conducted in triplicate.

P. mirabilis Biofilm

The bacterium P. mirabilis ATCC 29906 was also tested for the development of 3, 7, and 14 day-old biofilms on the surfaces of both control and SNAP impregnated silicone Foley catheter segments. Growth conditions were the same as those used for S. epidermidis biofilms. Procedures to assess cell viability were also performed as described above for S. epidermidis. For the second studies with P. mirabilis, the SNAP catheter segments were presoaked for 24 hours before being placed in the bioreactor. All experiments were conducted in triplicate.

Biofilm Imaging for Example 2

Control and SNAP impregnated catheter segments designated for fluorescent imaging were stained with LIVE/DEAD BacLight Bacterial Viability kit (L7012, Invitrogen, Carlsbad, Calif.) according to its instructions. Fluorescent images were acquired with an inverted fluorescence microscope (Olympus 1X71, Center Valley, Pa.) equipped with Fluorescence Illumination System (X-Cite 120, EXFO) and filters for SYTO-9 (excitation=488 nm/emission=520 nm) and Propidium Iodide (excitation=535 nm/emission=617 nm) fluorescence. Images were obtained using an oil immersion 60× objective lens. Only the fluorescent micrographs of control Foley catheters surfaces upon which P. mirabilis had been grown for 14 days were taken with a 10× objective lens due to the high biofilm biomass on these pieces of tubing. For these catheters, the use of the 60× objective lens would have impeded obtaining a clear focus of the biofilm layer on the catheter surfaces). All experiments were conducted in triplicate, and different surface areas of the catheter segments were randomly chosen for imaging.

Statistical Analysis for Example 2

Data for all of the experiments are expressed as mean±SEM (standard error of the mean). A comparison of means was performed using Student's t-test to analyze whether there was a statistical difference between the data for SNAP impregnated versus control silicone Foley catheter materials. Values of p<0.05 were considered statistically significant and graphically illustrated with a*.

NO Release from SNAP-Impregnated Silicone Foley Catheters

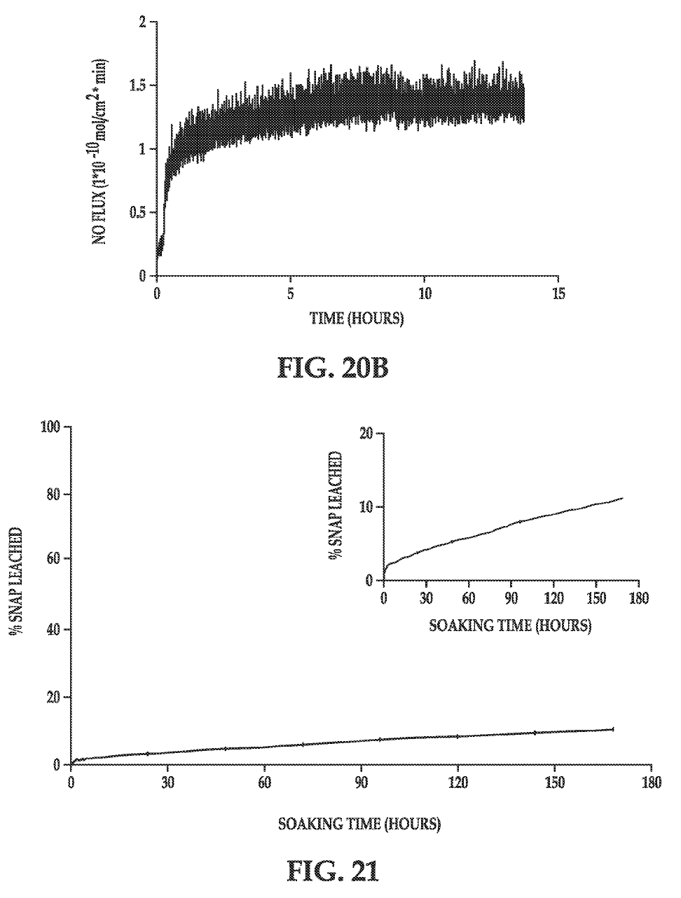

The total SNAP amount loaded into Foley catheters segments by the solvent swelling/impregnation method was found to be 5.43±0.15 wt % (n=3) using photolysis to emit the entire payload of NO in a reasonable time frame. Upon placing the dried tubing segments loaded with SNAP into PBS buffer at 37° C., SNAP decomposition within the walls of the tubing commenced due to the combination of both the presence of moisture and elevated temperature. The NO release data in FIG. 20A clearly show that the SNAP impregnated catheter tubing can release NO at a flux between 1.4 and 0.8 ×10⁻¹⁰ mol min⁻¹ cm⁻² for at least 30 days. For this experiment, both the outer and the inner wall surfaces of the pieces of tubing were utilized to calculate flux levels.

The NO release in the nitrogen-purged solution phase using the NOA was analyzed. As shown in FIG. 20B, the steady-state NO release profile, at any given time point for testing the silicone tubing, was relatively stable after about 30 minutes.

From the data in FIG. 20A, it is clear that the catheter tubing displayed an initial burst of NO during the first hours of soaking on day 1. This was likely due to the rapid reaction and leaching of SNAP present in the outermost surface layers of the silicone tubing. The long-term NO release data confirms that the swelling/impregnation method disclosed herein, conducted at room temperature, allows for the successful incorporation of SNAP within the polymer, and that NO release from the incorporated SNAP can last for at least 1 month at near constant flux levels over this period. This demonstrates the clear advantage of using the solvent swelling/impregnation approach over extruding the polymer with the NO donor present, since the latter would completely degrade the SNAP (loss in NO) due to the elevated extrusion temperatures.

SNAP Leaching from the Catheter Surface

To ensure that SNAP leaching from the catheter polymeric surface was primarily limited to the first day of soaking due to rapid water uptake at the outermost surface of the silicone rubber, leaching studies were conducted using UV-Vis absorbance spectroscopy over the first 7 days of soaking. At various time points, the UV-Vis spectra of aliquots of the PBS soaking solution were assayed for SNAP concentration. Since the total SNAP amount loaded into these 1 cm long catheter sections had previously been quantified, it was determined that only 3.51±0.04% of the total loaded SNAP leached from the catheter segment on the first day of soaking (see FIG. 21). Minimal amounts of SNAP continued to leach from the Foley catheters during the subsequent days of soaking. Indeed, as shown in the inset of FIG. 21, only 10.92±0.05% of the total SNAP was lost during the first week (168 hours).

N-acetyl-penicillamine (NAP) and/or NAP disulfide may also leach from the catheter surface. NAP may be used to treat mercury and other heavy metal poisoning, and thus its leaching has minimal toxicity concerns.

SNAP impregnated Foley catheters anti-biofilm properties against S. epidermidis

Catheterization is a principle risk factor for bacterial adhesion, colonization and subsequent infection. As such, the antimicrobial properties of the SNAP impregnated silicone Foley catheter segments with bacterial strains known to be associated with CAUTIs were examined.

S. epidermidis is a species associated with both intravascular and urinary infections. S. epidermidis represents a commensal inhabitant of healthy mucosal microflora, and it has the capacity to form high-biomass biofilms and colonize biomaterials. Biofilm-forming clinical strains of S. epidermidis, isolated from urinary tract infections, are significantly more resistant to a wide array of antibiotic treatments (e.g., ampicillin, ciprofloxacin, gentamicin, levofloxacin) than non-biofilm producing strains. The biofilm extracellular polysaccharide substance (EPS) may also represents a physical and chemical barrier to antibiotics. The polymeric matrices of biofilms may retard the penetration rate of antibiotics enough to induce the expression of genes that mediate antibiotic resistance. Low susceptibility to antibiotics may also be attributed by the metabolic state of microorganisms in a biofilm. As the cells located within the biofilm experience nutrient limitation, this condition can result in a stationary phase-like dormancy, thus influencing biofilm resistance to antibiotics as compared to bacteria in a planktonic stage. For at least these reasons, S. epidermidis was used in these experiments.

To simulate the development of bacterial biofilms on the surface of urinary Foley catheters, a CDC biofilm reactor was utilized. This reactor enables growth of mature biofilms and generates high shear force (flow-related turbulence) and is a standardized model to replicate urinary tract conditions in vivo. The total viable S. epidermidis adhered on the catheter tubing surface was determined after growing biofilms for either 3, 7, or 14 days at 37° C.

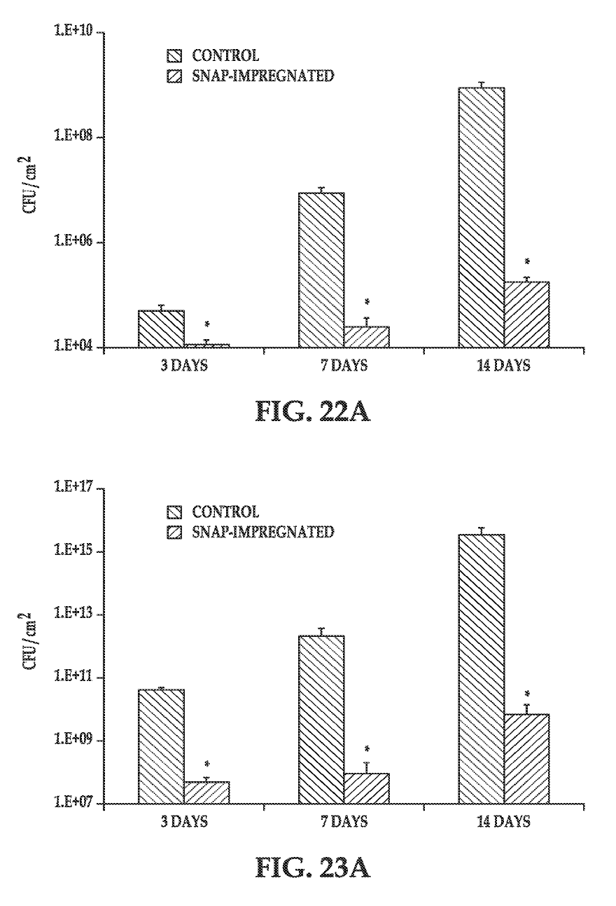

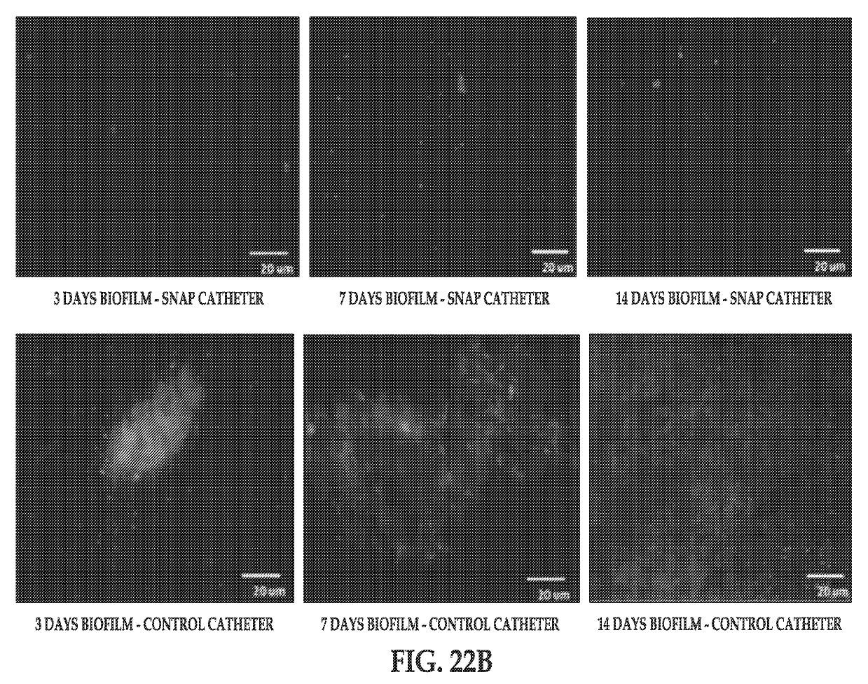

FIG. 22A shows the plate count data for 3 day old, 7 day old and 14 day old S. epidermidis biofilms on the surface of the SNAP impregnated catheter tubing and on the surface of the control tubing. As depicted in FIG. 22A, the plate count data for 3 day old S. epidermidis biofilms shows that the viable bacteria attached on the surface of the SNAP impregnated catheter tubing was about 50% less than on the controls (n=3, p value=0.014). A possible explanation for observing only a moderate reduction in cell viability may be that the bacteria had not yet developed into a mature biofilm, but were still in a micro-colony growth stage. At 3 days, the bacterial counts on the surfaces of the control catheters were relatively low, and thus a high percentage difference between the SNAP-doped and control catheters was not observed. However, the plate count data for 7 day S. epidermidis biofilms exhibited a significant 2.5 logarithmic unit difference in viable bacteria between control and NO releasing (i.e., SNAP impregnated) catheter segments (n=3, p value=0.015). Similarly, when S. epidermidis biofilms were grown for 14 days and reached a matured growth stage, the reduction in adhered viable bacteria on SNAP impregnated catheters compared to controls is even more pronounced, with a 3.7 logarithmic unit difference (n=3, p value=0.013). It is clear from the data shown in FIG. 22A that that the SNAP impregnated catheter segments can progressively reduce formation of S. Epidermidis biofilms.

The plate count data was substantiated by fluorescence imaging data. FIG. 22B illustrates black and white representations of the original fluorescence images that were taken. As shown in FIG. 22B, it is observed that while biofilm thickness and surface coverage increases with incubation time on control catheters, biofilm thickness and surface coverage remain relatively low over time on the surfaces of the SNAP impregnated catheters.

The results shown in FIGS. 22A and 22B illustrate that the SNAP impregnated silicone Foley catheters are capable of significantly limiting the viability of a prototypic biofilm-forming bacterium (S. epidermid/is). As such, the SNAP impregnated silicone Foley catheters disclosed herein may be used to successfully prevent nosocomial urinary infections.

SNAP Impregnated Foley Catheters Anti-Biofilm Properties Against P. mirabilis

The same experiments performed with S. epidermidis were performed with P. mirabilis, which is the primary bacterium associated with complicated urinary tract infection. This urease positive bacterial strain is also well known for its ability to establish mature crystalline biofilms. This bacterium elevates urine pH by catalyzing the conversion of urea into ammonia, inducing calcium/magnesium phosphates to precipitate from urine and become incorporated within the biofilm. For at least these reasons, P. mirabilis was used in these experiments.

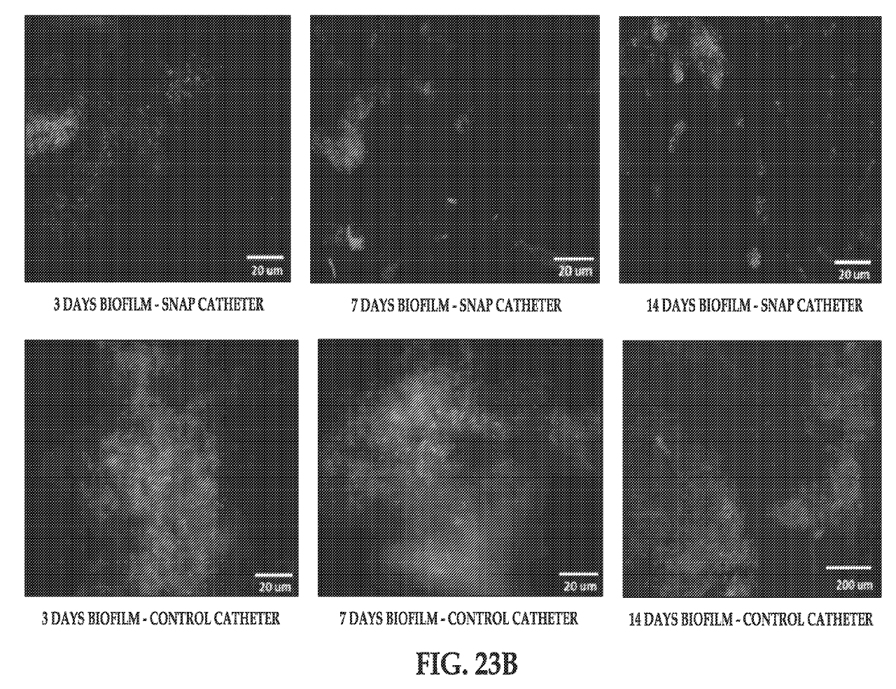

FIG. 23A shows the plate count data for 3 day old, 7 day old and 14 day old P. mirabilis biofilms on the surface of the SNAP impregnated catheter tubing and on the surface of the control tubing. As shown in FIG. 23A, the results for 3 day biofilms already show a 3 logarithmic unit difference in cell viability between the SNAP impregnated and control catheter segments (n=3, p value=0.02). Although the growth period is rather short, these biofilms had developed to a relatively mature stage.

Comparing FIGS. 22A and 23A, the plate count data (at 3 days) of the control catheter segments for the P. mirabilis experiments were significantly higher than the S. epidermidis biofilms. These results are also supported by the black and white representation of the fluorescent micrographs, shown in FIG. 23B, that show significantly greater biomass than the S. epidermidis biofilms (see FIG. 22B). Without being bound to any theory, it is believed that that P. mirabilis, by expressing mannose-resistant/Proteus-like (MR/P) fimbriae and P. mirabilis fimbriae (PMF), may be able to more rapidly and efficiently adhere/colonize onto the abiotic surfaces. Similar observations and comparisons between P. mirabilis and S. epidermidis can also be made for 7 day and 14 day biofilms, and an even greater decrease in biofilm formation is observed for P. mirabilis compared to S. epidermidis for the SNAP impregnated catheter pieces (see FIGS. 22A and 23A). In FIG. 23A, the cell counts for the SNAP impregnated catheter tubing are 5 logarithmic units (at 7 days, n=3, p value=0.02) and 6 logarithmic units (at 14 days, n=3, p value=0.007) lower than those for controls. It FIG. 23A, it the difference in viable P. mirabilis cell counts between SNAP impregnated catheter pieces and controls increases with biofilm growth time, indicating that the SNAP-doped catheters, by releasing NO, can progressively reduce biofilm development. This finding is further corroborated by the fluorescent imaging data (black and white representations of which are shown in FIG. 23B). FIG. 23B shows that bacterial surface coverage on the SNAP impregnated catheters segments is noticeably less than the controls. These results demonstrate that NO releasing SNAP impregnated Foley catheters may be suitable for preventing catheter surface encrustation by crystalline biofilm-forming bacterial species.

The data set forth herein with P. mirabilis illustrate that a synthetic NO-donor, with extremely stable NO release at low/non-toxic fluxes, is capable of preventing P. mirabilis mature biofilm formation. Other systems and/or methods have been used in attempts to prevent P. mirabilis mature biofilm formation. In some instances, biofilm inhibition had been observed only against non-mature biofilm communities. In other instances, the methods have not been successful for several reasons, such as instability and decay of the NO donor species, no protection against gram-negative bacteria, and antibiotic tolerance exhibited by the bacteria.

P. mirabilis Anti-Biofilm Study after Pre-Soaking SNAP Catheters for 24 Hours for Example 2

Chemiluminescence data, via the NOA, shows that the SNAP impregnated catheters display an initial burst of NO during the first day of soaking at 37° C., which is due to the thermal decomposition and the diffusion of SNAP out of the polymeric surface of the catheters. The bactericidal efficacies of NO releasing materials improve with increasing initial NO flux. To investigate non-toxic NO flux thresholds in urinary tract tissues, this experiment aimed for SNAP impregnated catheters to generate NO within ranges similar to those released by healthy endothelial cells (0.5-4 ×10⁻¹⁰ mol min⁻¹ cm⁻²). To ensure that SNAP impregnated catheters' antibiofilm properties were not due to the bactericidal effect of an initial high NO flux (4.5 ×10⁻¹⁰ mol min⁻¹ cm⁻²) during the initial hours of anti-bacterial studies in the bioreactor, additional experiments were conducted in which the SNAP catheters were first pre-soaked in PBS at 37° C. for 24 hours.

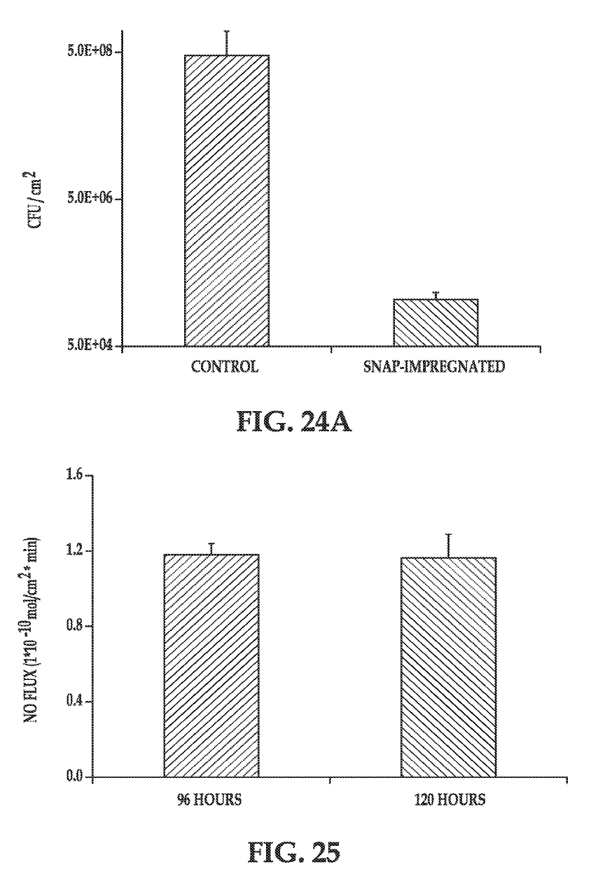

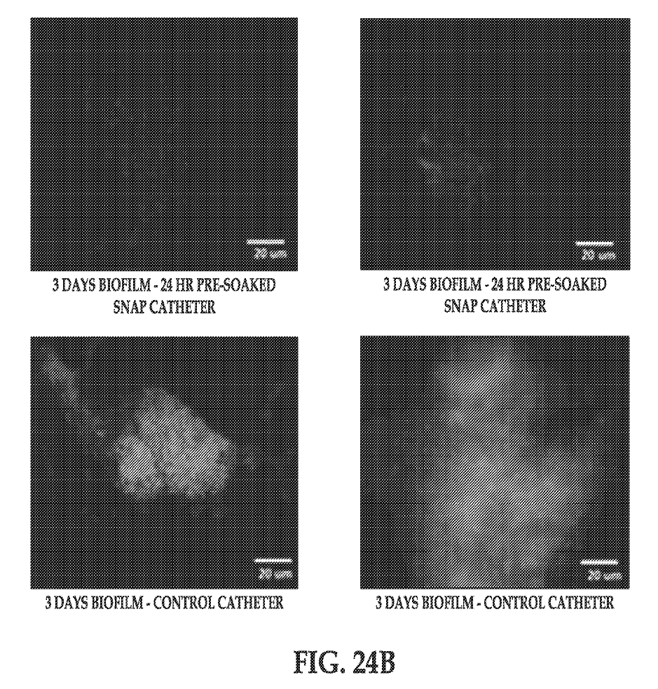

After this pre-soaking step, 3 day P. mirabilis anti-biofilms studies were conducted exactly as described above. FIG. 24A illustrates the plate count data of viable bacteria adhered on the catheters' surfaces. This data clearly illustrates that there is more than 3 logarithmic units difference between controls and pre-soaked SNAP impregnated catheters (n=6, p value=0.010). As such, in previous experiments, the initial release of high NO levels during day 1 was not the major factor contributing to the anti-biofilm properties of SNAP impregnated catheters. Indeed, even when SNAP impregnated catheters are pre-soaked for 24 hours before the start of the biofilm experiment, a significant reduction in viable cell counts can be observed for NO releasing catheters as compared to those for controls. These results are also supported by fluorescent images showing that bacterial surface coverage of pre-soaked SNAP impregnated catheters is noticeably less than the controls (black and white representations of the fluorescent images are shown in FIG. 24B).

SNAP Impregnated Foley Catheters NO Releasing Properties Post P. mirabilis Anti-Biofilm Experiment

To ensure that the SNAP impregnated catheter pieces can still release NO at a flux>0.5 ×10⁻¹⁰ mol cm⁻² near the end of the antibiofilm experiments, the NO releasing profile of three SNAP catheter segments, pre-soaked in PBS prior to the beginning of a 3 day P. mirabilis biofilm experiment, were tested via the NOA. As shown in FIG. 25, the SNAP impregnated catheter segments can still release NO at a flux >1 ×10⁻¹⁰ mol min⁻¹ cm⁻² at the end of the biofilm experiment (96 hours post initial soaking step) (FIG. 25, n=3).

Further, the NO releasing properties of the same catheter segments were then also tested on the following day, and the NO flux remained relatively constant (120 hours post initial soaking step) (FIG. 25, n=3). These results provide further evidence that the antibiofilm activity of SNAP impregnated catheters is due to the steady NO release properties of these novel biomedical devices.

Conclusions for Example 2

The data for Example 2 demonstrates that an FDA approved silicone Foley catheter material can be impregnated with SNAP via embodiments of a novel solvent swelling method, and that the resulting catheters can release a relatively steady NO flux at their surfaces for 30 days. Since no extrusion process is required for the incorporation of the NO donor/antimicrobial agent into the catheter walls with little or no chemical degradation of the SNAP, the catheters should maintain functionality with stable NO release capability.

The SNAP impregnated catheter tubings also exhibit significant anti-biofilm properties using two bacteria strains (S. epidermidis and P. mirabilis) that are responsible for high rates of nosocomial urinary catheter associated infections.

Furthermore, the preliminary assessment of the toxicity of SNAP impregnated catheter tubing was provided by Wuxi AppTec Inc. (St. Paul, Minn.) using ISO-based GLP biocompatibility studies. The catheter tubing received the safest scores possible (0) for in vitro toxicity testing (0-1=safe, 3-4=toxic) on L-929 Mouse Fibroblast Cells (extracts were taken from the catheter pieces stored for 24 hours at 37° C. in Eagle's Minimal Essential Media), as well as for in vivo testing in mice (extracts were taken from the catheter pieces stored for 72 hours at 37° C. in both saline and sesame oil). Hence, it is believed that the SNAP impregnated Foley urinary catheters provide a cost effective and suitable approach to dramatically reduce the frequency of nosocomial CAUTIs.

In one embodiment of the novel method, the soaking of a polymer material, such as a cross-linked tube, catheter, or slab, having one or more dedicated inner lumens can be conducted through the inner lumen/s of the polymer material to avoid exposure of the outside surface of the polymer material to the solvent or impregnation solution. In one embodiment, this is accomplished by filling a dedicated inner lumen with the NO donor impregnation solution at concentrations between 20 mg/mL and 120 mg/mL, depending on the desired NO release profile, and allowing the polymer material to soak and inner impregnate for 2 to 12 hours in the dark. Next, the treated catheters may be quickly rinsed with ethanol and air dried through the inner lumen, followed by drying in the dark for 48 hours. These embodiments of the novel method allows for a smoother polymer material outer surface where live tissue may make contact.

It is to be understood that the ranges provided herein include the stated range and any value or sub-range within the stated range. For example, a range of about 0.2 ×10⁻¹⁰ mol cm⁻² min⁻¹ to about 20 ×10⁻¹⁰ mol cm⁻² min⁻¹ should be interpreted to include not only the explicitly recited limits of 0.2 ×10⁻¹⁰ mol cm⁻² min⁻¹ to about 20 ×10⁻¹⁰ mol cm⁻² min⁻¹, but also to include individual values there between, such as 1 ×10⁻¹⁰ mol cm⁻² min⁻¹, 14.5 ×10⁻¹⁰ mol cm⁻² min⁻¹, etc., as well as sub-ranges therebetween, such as from 0.75 ×10⁻¹⁰ mol cm⁻² min⁻¹ to about 17×10⁻¹⁰ mol cm⁻² min⁻¹, from about 5 ×10⁻¹⁰ mol cm⁻² min⁻¹ to about 15 ×10⁻¹⁰ mol cm⁻² min⁻¹, etc. Furthermore, when “about” or “approximately” or the like is/are utilized to describe a value, this is meant to encompass minor variations (up to +/−10%) from the stated value.

Reference throughout the specification to “one example”, “another example”, “an example”, and so forth, means that a particular element (e.g., feature, structure, and/or characteristic) described in connection with the example is included in at least one example described herein, and may or may not be present in other examples. In addition, it is to be understood that the described elements for any example may be combined in any suitable manner in the various examples unless the context clearly dictates otherwise.

Furthermore, in describing and claiming the examples disclosed herein, the singular forms “a”, “an”, and “the” include plural referents unless the context clearly dictates otherwise.

While several examples have been described in detail, it will be apparent to those skilled in the art that the disclosed examples may be modified. Therefore, the foregoing description is to be considered non-limiting.

1. A method for making a NO-releasing polymeric composition, comprising the steps of:
dissolving a discrete RSNO adduct in a solvent to form a discrete RSNO adduct solution, wherein the solvent is selected from the group consisting of tetrahydrofuran, chloroform, methylene chloride, cyclohexanone, and combinations thereof; and
soaking a polymer material in the discrete RSNO adduct solution for a predetermined time to swell the polymer material and impregnate the polymer material with the discrete RSNO adduct.

2. The method as defined in claim 1 wherein the polymer material is selected from the group consisting of a siloxane-based polyurethane elastomer, silicone rubber, and a thermoplastic silicone-polycarbonate-urethane, and wherein the discrete RSNO adduct is S-nitroso-N-acetylpenicillamine.

3. The method as defined in claim 1 wherein the dissolving and the soaking are accomplished at room temperature ranging from about 17° C. to about 24° C.

4. The method as defined in claim 1 wherein the dissolving is accomplished by:
adding S-nitroso-N-acetylpenicillamine to the solvent; and
stirring the solution for a predetermined time.

5. The method as defined in claim 1, further comprising completely immersing the polymer material in the discrete RSNO adduct solution prior to the soaking.

6. The method as defined in claim 1 wherein the predetermined time is about 24 hours, and wherein the soaking is accomplished in darkness.

7. The method as defined in claim 1 wherein after the soaking for the predetermined time, the method further comprises drying the impregnated polymer material in darkness.

8. A method for making an NO-releasing polymeric composition, comprising the steps of:
dissolving a discrete RSNO adduct in a solvent to form a discrete RSNO adduct solution; and
soaking a polymer material, wherein the polymer material is selected from the group consisting of a siloxane-based polyurethane elastomer, silicone rubber, and a thermoplastic silicone-polycarbonate-urethane, in the discrete RSNO adduct solution for a predetermined time to swell the polymer material and impregnate the polymer material with the discrete RSNO adduct.

9. The method as defined in claim 8 wherein the discrete RSNO adduct is S-nitroso-N-acetylpenicillamine.

10. The method as defined in claim 8 wherein the dissolving and the soaking are accomplished at room temperature ranging from about 17° C. to about 24° C.

11. The method as defined in claim 8 wherein the dissolving is accomplished by:
adding S-nitroso-N-acetylpenicillamine to the solvent, wherein the solvent is selected from the group consisting of tetrahydrofuran, chloroform, methylene chloride, cyclohexanone, and combinations thereof; and
stirring the solution.

12. The method as defined in claim 8, wherein soaking involves completely immersing the polymer material in the discrete RSNO adduct solution.

13. The method as defined in claim 8 wherein the time is about 24 hours, and wherein the soaking is accomplished in darkness.

14. The method as defined in claim 8 wherein after the soaking for the predetermined time, the method further comprises drying the impregnated polymer material in darkness.

15. A method for making a NO-releasing polymeric composition, comprising the steps of:
dissolving a NO donor molecule in a solvent to form a NO donor solution; and
soaking a polymer material with the NO donor solution for a predetermined time, wherein the polymer material is a tube or catheter.

16. The method as defined in claim 15 wherein the soaking of the polymer material is conducted only through an inner lumen of the polymer material.

17. The method as defined in claim 15 wherein the resulting NO-releasing polymeric composition releases NO at a near constant flux for at least 12 hours.

18. The method as defined in claim 15 wherein the concentration of the NO donor in the NO donor solution is 10 mg/mL-128 mg/mL.

19. The method as defined in claim 15 wherein the solubility of the NO donor in the solvent is at least 10 mg/mL.

20. A NO-releasing polymeric composition made according to the method as defined in claim 15.
